(19) United States
(12) Patent Application Publication
(10) Pub. No.: US 2023/0233713 A1
(43) Pub. Date: Jul. 27, 2023

(54) A HYPERTENSION ANIMAL MODEL AND METHODS OF USE

(71) Applicant: Board of Supervisors of Louisiana State University and Agricultural and Mechanical College, Baton Rouge, LA (US)

(72) Inventors: David J. Lefer, Saint Bernard, LA (US); Thomas Sharp, Baton Rouge, LA (US); Traci Goodchild, Baton Rouge, LA (US)

(21) Appl. No.: 18/012,579

(22) PCT Filed: Jun. 25, 2021
(86) PCT No.: PCT/US2021/039130

§ 371 Date: Dec. 22, 2022

Related U.S. Application Data
(60) Provisional application No. 63/044,865, filed on Jun. 26, 2020.
(60) Provisional application No. 63/104,678, filed on Oct. 23, 2020.

(51) Int. Cl. A61K 49/00; A23K 20/105; A23K 20/137; A23K 20/163; A23K 20/158; A23K 20/147; A23K 50/30; A61K 31/573; A61K 31/223; A61K 38/08; A61P 9/02

(52) U.S. Cl. CPC A61K 49/0008; A23K 20/105; A23K 20/137; A23K 20/163; A23K 20/158; A23K 20/147; A23K 50/30; A61K 31/573; A61K 31/223; A61K 38/085; A61P 9/02

(57) ABSTRACT

The invention is directed to a hypertension animal model, and methods of using the same to induce heart failure with preserved ejection fraction (HFpEF) responses.

This application claims priority from U.S. Provisional Application No. 63/044,865, filed on Jun. 26, 2020, and U.S. Provisional Application No. 63/104,678, filed on Oct. 23, 2020, the entire contents of each of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

The invention is directed to a hypertension animal model, and methods of using the same to induce heart failure with preserved ejection fraction (HFpEF) responses.

### BACKGROUND OF THE INVENTION

Heart failure (HF) is a chronic and progressive condition where the heart muscle is unable to pump enough blood to meet the body's requirements for blood and oxygen. HF is the most prevalent form of cardiac disease in the US. There are two types of HF: (1) HF with reduced ejection fraction (HFrEF) where the heart does not contract effectively; and (2) HF with preserved ejection fraction (HFpEF) where the heart contractility is normal but is unable to fill properly with blood during the diastolic (filling) phase.

### SUMMARY OF THE INVENTION

An aspect of the invention is directed to an animal HFpEF composition. In one embodiment the HFpEF composition comprises a hypertensive agent formulated in a dose of about 5 mg/kg to about 100 mg/kg; and an animal chow [HFpEF Diet] comprising about a 50%-50% (wt/wt) proportion of standard diet and custom diet. In one embodiment, the hypertensive agent comprises a pellet or continuous infusion. In another embodiment, the hypertensive agent comprises 11-deoxycorticosterone acetate (DOCA), N(gamma)-nitro-L-arginine methyl ester (L-NAME), or Angiotensin II (ANG II). In one embodiment, the hypertensive agent comprises a pellet or continuous infusion. In one embodiment, the standard diet comprises about 33% carbohydrate, about 3.9% fat, about 16.1% protein, about 0% cholesterol, about 0.4% salt, about 2.3 kcal/g energy density, or a combination of the standard diet components described herein. In one embodiment, the custom diet comprises about 39.8% carbohydrate, about 40% to about 44.1% fat, about 16.2% protein, about 2% cholesterol, about 0.5% to about 0.7% sodium cholate, about 4.01 kcal/g energy density, or a combination of the custom diet components described herein. In one embodiment, the custom diet further comprises about 4% salt. In one embodiment, the custom diet further comprises carbohydrates that comprise about 17.8% fructose. In some embodiments, DOCA is formulated in a dose of about 25 mg/kg to about 100 mg/kg.

An aspect of the invention is directed to a method for inducing heart failure with preserved ejection fraction (HFpEF) in a laboratory animal. In one embodiment, the method comprises administering the HFpEF composition of any one of the animal HFpEF compositions described herein to a swine laboratory animal. In one embodiment, the hypertensive agent comprises 11-deoxycorticosterone acetate (DOCA), N(gamma)-nitro-L-arginine methyl ester (L-NAME), or Angiotensin II (ANG II). In one embodiment, the hypertensive agent comprises a pellet or continuous infusion. In one embodiment, the animal chow comprises about a 50%-50% (wt/wt) proportion of standard diet and custom diet, wherein the standard diet comprises about 33% carbohydrate, about 3.9% fat, about 16.1% protein, about 0% cholesterol, about 0.4% salt, about 2.3 kcal/g energy density, or a combination of the standard diet components described herein; and the custom diet comprises about 39.8% carbohydrate, about 40% to about 44.1% fat, about 16.2% protein, about 2% cholesterol, about 0.5% to about 0.7% sodium cholate, about 4.01 kcal/g energy density, or a combination of the custom diet components described herein. In embodiments, animal chow comprises an HFpEF diet. In one embodiment, the custom diet further comprises about 4% salt. In one embodiment, the custom diet further comprises carbohydrates that comprise about 17.8% fructose. In one embodiment, DOCA is formulated in a dose of about 25 mg/kg to about 100 mg/kg. In one embodiment, the DOCA pellet is administered subcutaneously or orally. In one embodiment, DOCA, L-NAME, or ANG II is administered subcutaneously, intravenously, transdermal, or orally. In one embodiment, the administering occurs from 30 days to 90 days to induce and maintain high blood pressure. In one embodiment, the blood pressure comprises an arterial systolic blood pressure ≥about 130 mmHg, an arterial diastolic blood pressure ≥about 90 mmHg, a mean arterial blood pressure ≥about 100 mmHg, a left ventricular end-diastolic pressure ≥about 14 mmHg, or a combination of these blood pressure readouts. In one embodiment, the blood pressure comprises an arterial systolic blood pressure ≥about 150 mmHg, an arterial diastolic blood pressure ≥about 110 mmHg, a mean arterial blood pressure ≥about 130 mmHg, a left ventricular end-diastolic pressure ≥about 14 mmHg, or a combination of these blood pressure readouts. In one embodiment, the blood pressure further comprises elevated exercise intolerance, and/or LV diastolic dysfunction (elevated E/e′). In one embodiment, the animal chow is provided daily as a total dry weight of about 900 grams. In one embodiment, the method further comprises providing water ad libitum to the animal. In one embodiment, the water comprises about 40 mg salt per 1 liter. In one embodiment, the animal chow and water are administered for at least 20 weeks. In one embodiment, the animal is a swine laboratory animal. In one embodiment, the swine animal is a Gottingen Minipig®.

An aspect of the invention is directed to a method for inducing chronic hypertension in a laboratory animal. In one embodiment, the method comprises administering the composition of any one of the animal HFpEF compositions described herein to a swine laboratory animal. In one embodiment, the hypertensive agent comprises 11-deoxycorticosterone acetate (DOCA), N(gamma)-nitro-L-arginine methyl ester (L-NAME), or Angiotensin II (ANG II). In one embodiment, the hypertensive agent comprises a pellet or continuous infusion. In one embodiment, the animal chow comprises about a 50%-50% (wt/wt) proportion of standard diet and custom diet, wherein the standard diet comprises about 33% carbohydrate, about 3.9% fat, about 16.1% protein, about 0% cholesterol, about 0.4% salt, about 2.3 kcal/g energy density, or a combination of the standard diet components described herein; and the custom diet comprises about 39.8% carbohydrate, about 40% to about 44.1% fat, about 16.2% protein, about 2% cholesterol, about 0.5% to about 0.7% sodium cholate, about 4.01 kcal/g energy density, or a combination of the custom diet components described herein. In one embodiment, the custom diet further comprises about 4% salt. In one embodiment, the custom diet further comprises carbohydrates that comprise about 17.8% fructose. In one embodiment, DOCA is formulated in a dose of about 25 mg/kg to about 100 mg/kg. In one embodiment, the DOCA pellet is administered subcutaneously or orally. In one embodiment, DOCA, L-NAME, or ANG II is administered subcutaneously, intravenously, transdermal, or orally. In one embodiment, the administering occurs from 30 days to 90 days to induce and maintain high blood pressure. In one embodiment, the blood pressure comprises an arterial systolic blood pressure ≥about 130 mmHg, an arterial diastolic blood pressure ≥about 90 mmHg, a mean arterial blood pressure ≥about 100 mmHg, a left ventricular end-diastolic pressure ≥about 14 mmHg, or a combination of these blood pressure readouts. In one embodiment, the blood pressure comprises an arterial systolic blood pressure ≥about 150 mmHg, an arterial diastolic blood pressure ≥about 110 mmHg, a mean arterial blood pressure ≥about 130 mmHg, a left ventricular end-diastolic pressure ≥about 14 mmHg, or a combination of these blood pressure readouts. In one embodiment, the blood pressure further comprises elevated exercise intolerance, and/or LV diastolic dysfunction (elevated E/e′). In one embodiment, the animal chow is provided daily as a total dry weight of about 900 grams. In one embodiment, the method further comprises providing water ad libitum to the animal. In one embodiment, the water comprises about 40 mg salt per 1 liter. In one embodiment, the animal chow and water are administered for at least 20 weeks. In one embodiment, the animal is a swine laboratory animal. In one embodiment, the swine animal is a Gottingen Minipig®.

An aspect of the invention is directed to a method for inducing atherosclerosis in a laboratory animal. In one embodiment, the method comprises administering the composition of any one of the animal HFpEF compositions described herein to a swine laboratory animal. In one embodiment, the hypertensive agent comprises 11-deoxycorticosterone acetate (DOCA), N(gamma)-nitro-L-arginine methyl ester (L-NAME), or Angiotensin II (ANG II). In one embodiment, the hypertensive agent comprises a pellet or continuous infusion. In one embodiment, the animal chow comprises about a 50%-50% (wt/wt) proportion of standard diet and custom diet, wherein the standard diet comprises about 33% carbohydrate, about 3.9% fat, about 16.1% protein, about 0% cholesterol, about 0.4% salt, about 2.3 kcal/g energy density, or a combination of the standard diet components described herein; and the custom diet comprises about 39.8% carbohydrate, about 40% to about 44.1% fat, about 16.2% protein, about 2% cholesterol, about 0.5% to about 0.7% sodium cholate, about 4.01 kcal/g energy density, or a combination of the custom diet components described herein. In one embodiment, the custom diet further comprises about 4% salt. In one embodiment, the custom diet further comprises carbohydrates that comprise about 17.8% fructose. In one embodiment, DOCA is formulated in a dose of about 25 mg/kg to about 100 mg/kg. In one embodiment, the DOCA pellet is administered subcutaneously or orally. In one embodiment, DOCA, L-NAME, or ANG II is administered subcutaneously, intravenously, transdermal, or orally. In one embodiment, the administering occurs from 30 days to 90 days to induce and maintain high blood pressure. In one embodiment, the blood pressure comprises an arterial systolic blood pressure ≥about 130 mmHg, an arterial diastolic blood pressure ≥about 90 mmHg, a mean arterial blood pressure ≥about 100 mmHg, a left ventricular end-diastolic pressure ≥about 14 mmHg, or a combination of these blood pressure readouts. In one embodiment, the blood pressure comprises an arterial systolic blood pressure ≥about 150 mmHg, an arterial diastolic blood pressure ≥about 110 mmHg, a mean arterial blood pressure ≥about 130 mmHg, a left ventricular end-diastolic pressure ≥about 14 mmHg, or a combination of these blood pressure readouts. In one embodiment, the blood pressure further comprises elevated exercise intolerance, and/or LV diastolic dysfunction (elevated E/e′). In one embodiment, the animal chow is provided daily as a total dry weight of about 900 grams. In one embodiment, the method further comprises providing water ad libitum to the animal. In one embodiment, the water comprises about 40 mg salt per 1 liter. In one embodiment, the animal chow and water are administered for at least 20 weeks. In one embodiment, the animal is a swine laboratory animal. In one embodiment, the swine animal is a Gottingen Minipig®.

An aspect of the invention is directed to a method for inducing vascular injury and endothelial dysfunction in a laboratory animal. In one embodiment, the method comprises administering the composition of any one of the animal HFpEF compositions described herein to a swine laboratory animal. In one embodiment, the hypertensive agent comprises 11-deoxycorticosterone acetate (DOCA), N(gamma)-nitro-L-arginine methyl ester (L-NAME), or Angiotensin II (ANG II). In one embodiment, the hypertensive agent comprises a pellet or continuous infusion. In one embodiment, the animal chow comprises about a 50%-50% (wt/wt) proportion of standard diet and custom diet, wherein the standard diet comprises about 33% carbohydrate, about 3.9% fat, about 16.1% protein, about 0% cholesterol, about 0.4% salt, about 2.3 kcal/g energy density, or a combination of the standard diet components described herein; and the custom diet comprises about 39.8% carbohydrate, about 40% to about 44.1% fat, about 16.2% protein, about 2% cholesterol, about 0.5% to about 0.7% sodium cholate, about 4.01 kcal/g energy density, or a combination of the custom diet components described herein. In one embodiment, the custom diet further comprises about 4% salt. In one embodiment, the custom diet further comprises carbohydrates that comprise about 17.8% fructose. In one embodiment, DOCA is formulated in a dose of about 25 mg/kg to about 100 mg/kg. In one embodiment, the DOCA pellet is administered subcutaneously or orally. In one embodiment, DOCA, L-NAME, or ANG II is administered subcutaneously, intravenously, transdermal, or orally. In one embodiment, the administering occurs from 30 days to 90 days to induce and maintain high blood pressure. In one embodiment, the blood pressure comprises an arterial systolic blood pressure ≥about 130 mmHg, an arterial diastolic blood pressure ≥about 90 mmHg, a mean arterial blood pressure ≥about 100 mmHg, a left ventricular end-diastolic pressure ≥about 14 mmHg, or a combination of these blood pressure readouts. In one embodiment, the blood pressure comprises an arterial systolic blood pressure ≥about 150 mmHg, an arterial diastolic blood pressure ≥about 110 mmHg, a mean arterial blood pressure ≥about 130 mmHg, a left ventricular end-diastolic pressure ≥about 14 mmHg, or a combination of these blood pressure readouts. In one embodiment, the blood pressure further comprises elevated exercise intolerance, and/or LV diastolic dysfunction (elevated E/e′). In one embodiment, the animal chow is provided daily as a total dry weight of about 900 grams. In one embodiment, the method further comprises providing water ad libitum to the animal. In one embodiment, the water comprises about 40 mg salt per 1 liter. In one embodiment, the animal chow and water are administered for at least 20 weeks. In one embodiment, the animal is a swine laboratory animal. In one embodiment, the swine animal is a Gottingen Minipig®.

An aspect of the invention is directed to a method for inducing peripheral vascular disease in a laboratory animal. In one embodiment, the method comprises administering the composition of any one of the animal HFpEF compositions described herein to a swine laboratory animal. In one embodiment, the hypertensive agent comprises 11-deoxycorticosterone acetate (DOCA), N(gamma)-nitro-L-arginine methyl ester (L-NAME), or Angiotensin II (ANG II). In one embodiment, the hypertensive agent comprises a pellet or continuous infusion. In one embodiment, the animal chow comprises about a 50%-50% (wt/wt) proportion of standard diet and custom diet, wherein the standard diet comprises about 33% carbohydrate, about 3.9% fat, about 16.1% protein, about 0% cholesterol, about 0.4% salt, about 2.3 kcal/g energy density, or a combination of the standard diet components described herein; and the custom diet comprises about 39.8% carbohydrate, about 40% to about 44.1% fat, about 16.2% protein, about 2% cholesterol, about 0.5% to about 0.7% sodium cholate, about 4.01 kcal/g energy density, or a combination of the custom diet components described herein. In one embodiment, the custom diet further comprises about 4% salt. In one embodiment, the custom diet further comprises carbohydrates that comprise about 17.8% fructose. In one embodiment, DOCA is formulated in a dose of about 25 mg/kg to about 100 mg/kg. In one embodiment, the DOCA pellet is administered subcutaneously or orally. In one embodiment, DOCA, L-NAME, or ANG II is administered subcutaneously, intravenously, transdermal, or orally. In one embodiment, the administering occurs from 30 days to 90 days to induce and maintain high blood pressure. In one embodiment, the blood pressure comprises an arterial systolic blood pressure ≥about 130 mmHg, an arterial diastolic blood pressure ≥about 90 mmHg, a mean arterial blood pressure ≥about 100 mmHg, a left ventricular end-diastolic pressure ≥about 14 mmHg, or a combination of these blood pressure readouts. In one embodiment, the blood pressure comprises an arterial systolic blood pressure ≥about 150 mmHg, an arterial diastolic blood pressure ≥about 110 mmHg, a mean arterial blood pressure ≥130 mmHg, a left ventricular end-diastolic pressure ≥about 14 mmHg, or a combination of these blood pressure readouts. In one embodiment, the blood pressure further comprises elevated exercise intolerance, and/or LV diastolic dysfunction (elevated E/e′). In one embodiment, the animal chow is provided daily as a total dry weight of about 900 grams. In one embodiment, the method further comprises providing water ad libitum to the animal. In one embodiment, the water comprises about 40 mg salt per 1 liter. In one embodiment, the animal chow and water are administered for at least 20 weeks. In one embodiment, the animal is a swine laboratory animal. In one embodiment, the swine animal is a Gottingen Minipig®.

An aspect of the invention is directed to a method for inducing pulmonary hypertension in a laboratory animal. In one embodiment, the method comprises administering the composition of any one of the animal HFpEF compositions described herein to a swine laboratory animal. In one embodiment, the hypertensive agent comprises 11-deoxycorticosterone acetate (DOCA), N(gamma)-nitro-L-arginine methyl ester (L-NAME), or Angiotensin II (ANG II). In one embodiment, the hypertensive agent comprises a pellet or continuous infusion. In one embodiment, the animal chow comprises about a 50%-50% (wt/wt) proportion of standard diet and custom diet, wherein the standard diet comprises about 33% carbohydrate, about 3.9% fat, about 16.1% protein, about 0% cholesterol, about 0.4% salt, about 2.3 kcal/g energy density, or a combination of the standard diet components described herein; and the custom diet comprises about 39.8% carbohydrate, about 40% to about 44.1% fat, about 16.2% protein, about 2% cholesterol, about 0.5% to about 0.7% sodium cholate, about 4.01 kcal/g energy density, or a combination of the custom diet components described herein. In one embodiment, the custom diet further comprises about 4% salt. In one embodiment, the custom diet further comprises carbohydrates that comprise about 17.8% fructose. In one embodiment, DOCA is formulated in a dose of about 25 mg/kg to about 100 mg/kg. In one embodiment, the DOCA pellet is administered subcutaneously or orally. In one embodiment, DOCA, L-NAME, or ANG II is administered subcutaneously, intravenously, transdermal, or orally. In one embodiment, the administering occurs from 30 days to 90 days to induce and maintain high blood pressure. In one embodiment, the blood pressure comprises an arterial systolic blood pressure ≥about 130 mmHg, an arterial diastolic blood pressure ≥about 90 mmHg, a mean arterial blood pressure ≥about 100 mmHg, a left ventricular end-diastolic pressure ≥about 14 mmHg, or a combination of these blood pressure readouts. In one embodiment, the blood pressure comprises an arterial systolic blood pressure ≥about 150 mmHg, an arterial diastolic blood pressure ≥about 110 mmHg, a mean arterial blood pressure ≥about 130 mmHg, a left ventricular end-diastolic pressure ≥about 14 mmHg, or a combination of these blood pressure readouts. In one embodiment, the blood pressure further comprises elevated exercise intolerance, and/or LV diastolic dysfunction (elevated E/e′). In one embodiment, the animal chow is provided daily as a total dry weight of about 900 grams. In one embodiment, the method further comprises providing water ad libitum to the animal. In one embodiment, the water comprises about 40 mg salt per 1 liter. In one embodiment, the animal chow and water are administered for at least 20 weeks. In one embodiment, the animal is a swine laboratory animal. In one embodiment, the swine animal is a Gottingen Minipig®.

An aspect of the invention is directed to a method for inducing fatty liver (steatohepatitis) in a laboratory animal. In one embodiment, the method comprises administering the composition of any one of the animal HFpEF compositions described herein to a swine laboratory animal. In one embodiment, the hypertensive agent comprises 11-deoxycorticosterone acetate (DOCA), N(gamma)-nitro-L-arginine methyl ester (L-NAME), or Angiotensin II (ANG II). In one embodiment, the hypertensive agent comprises a pellet or continuous infusion. In one embodiment, the animal chow comprises about a 50%-50% (wt/wt) proportion of standard diet and custom diet, wherein the standard diet comprises about 33% carbohydrate, about 3.9% fat, about 16.1% protein, about 0% cholesterol, about 0.4% salt, about 2.3 kcal/g energy density, or a combination of the standard diet components described herein; and the custom diet comprises about 39.8% carbohydrate, about 40% to about 44.1% fat, about 16.2% protein, about 2% cholesterol, about 0.5% to about 0.7% sodium cholate, about 4.01 kcal/g energy density, or a combination of the custom diet components described herein. In one embodiment, the custom diet further comprises about 4% salt. In one embodiment, the custom diet further comprises carbohydrates that comprise about 17.8% fructose. In one embodiment, DOCA is formulated in a dose of about 25 mg/kg to about 100 mg/kg. In one embodiment, the DOCA pellet is administered subcutaneously or orally. In one embodiment, DOCA, L-NAME, or ANG II is administered subcutaneously, intravenously, transdermal, or orally. In one embodiment, the administering occurs from 30 days to 90 days to induce and maintain high blood pressure. In one embodiment, the blood pressure comprises an arterial systolic blood pressure ≥about 130 mmHg, an arterial diastolic blood pressure ≥about 90 mmHg, a mean arterial blood pressure ≥about 100 mmHg, a left ventricular end-diastolic pressure ≥about 14 mmHg, or a combination of these blood pressure readouts. In one embodiment, the blood pressure comprises an arterial systolic blood pressure ≥about 150 mmHg, an arterial diastolic blood pressure ≥110 mmHg, a mean arterial blood pressure ≥about 130 mmHg, a left ventricular end-diastolic pressure ≥about 14 mmHg, or a combination of these blood pressure readouts. In one embodiment, the blood pressure further comprises elevated exercise intolerance, and/or LV diastolic dysfunction (elevated E/e′). In one embodiment, the animal chow is provided daily as a total dry weight of about 900 grams. In one embodiment, the method further comprises providing water ad libitum to the animal. In one embodiment, the water comprises about 40 mg salt per 1 liter. In one embodiment, the animal chow and water are administered for at least 20 weeks. In one embodiment, the animal is a swine laboratory animal. In one embodiment, the swine animal is a Gottingen Minipig®.

An aspect of the invention is directed to a method for inducing chronic kidney disease or renal disease in a laboratory animal. In one embodiment, the method comprises administering the composition of any one of the animal HFpEF compositions described herein to a swine laboratory animal. In one embodiment, the hypertensive agent comprises 11-deoxycorticosterone acetate (DOCA), N(gamma)-nitro-L-arginine methyl ester (L-NAME), or Angiotensin II (ANG II). In one embodiment, the hypertensive agent comprises a pellet or continuous infusion. In one embodiment, the animal chow comprises about a 50%-50% (wt/wt) proportion of standard diet and custom diet, wherein the standard diet comprises about 33% carbohydrate, about 3.9% fat, about 16.1% protein, about 0% cholesterol, about 0.4% salt, about 2.3 kcal/g energy density, or a combination of the standard diet components described herein; and the custom diet comprises about 39.8% carbohydrate, about 40% to about 44.1% fat, about 16.2% protein, about 2% cholesterol, about 0.5% to about 0.7% sodium cholate, about 4.01 kcal/g energy density, or a combination of the custom diet components described herein. In one embodiment, the custom diet further comprises about 4% salt. In one embodiment, the custom diet further comprises carbohydrates that comprise about 17.8% fructose. In one embodiment, DOCA is formulated in a dose of about 25 mg/kg to about 100 mg/kg. In one embodiment, the DOCA pellet is administered subcutaneously or orally. In one embodiment, DOCA, L-NAME, or ANG II is administered subcutaneously, intravenously, transdermal, or orally. In one embodiment, the administering occurs from 30 days to 90 days to induce and maintain high blood pressure. In one embodiment, the blood pressure comprises an arterial systolic blood pressure ≥about 130 mmHg, an arterial diastolic blood pressure ≥about 90 mmHg, a mean arterial blood pressure ≥about 100 mmHg, a left ventricular end-diastolic pressure ≥about 14 mmHg, or a combination of these blood pressure readouts. In one embodiment, the blood pressure comprises an arterial systolic blood pressure ≥about 150 mmHg, an arterial diastolic blood pressure ≥about 110 mmHg, a mean arterial blood pressure ≥about 130 mmHg, a left ventricular end-diastolic pressure ≥about 14 mmHg, or a combination of these blood pressure readouts. In one embodiment, the blood pressure further comprises elevated exercise intolerance, and/or LV diastolic dysfunction (elevated E/e′). In one embodiment, the animal chow is provided daily as a total dry weight of about 900 grams. In one embodiment, the method further comprises providing water ad libitum to the animal. In one embodiment, the water comprises about 40 mg salt per 1 liter. In one embodiment, the animal chow and water are administered for at least 20 weeks. In one embodiment, the animal is a swine laboratory animal. In one embodiment, the swine animal is a Gottingen Minipig®.

An aspect of the invention is directed to a method for inducing obesity in a laboratory animal. In one embodiment, the method comprises administering the composition of any one of the animal HFpEF compositions described herein to a swine laboratory animal. In one embodiment, the hypertensive agent comprises 11-deoxycorticosterone acetate (DOCA), N(gamma)-nitro-L-arginine methyl ester (L-NAME), or Angiotensin II (ANG II). In one embodiment, the hypertensive agent comprises a pellet or continuous infusion. In one embodiment, the animal chow comprises about a 50%-50% (wt/wt) proportion of standard diet and custom diet, wherein the standard diet comprises about 33% carbohydrate, about 3.9% fat, about 16.1% protein, about 0% cholesterol, about 0.4% salt, about 2.3 kcal/g energy density, or a combination of the standard diet components described herein; and the custom diet comprises about 39.8% carbohydrate, about 40% to about 44.1% fat, about 16.2% protein, about 2% cholesterol, about 0.5% to about 0.7% sodium cholate, about 4.01 kcal/g energy density, or a combination of the custom diet components described herein. In one embodiment, the custom diet further comprises about 4% salt. In one embodiment, the custom diet further comprises carbohydrates that comprise about 17.8% fructose. In one embodiment, DOCA is formulated in a dose of about 25 mg/kg to about 100 mg/kg. In one embodiment, the DOCA pellet is administered subcutaneously or orally. In one embodiment, DOCA, L-NAME, or ANG II is administered subcutaneously, intravenously, transdermal, or orally. In one embodiment, the administering occurs from 30 days to 90 days to induce and maintain high blood pressure. In one embodiment, the blood pressure comprises an arterial systolic blood pressure ≥about 130 mmHg, an arterial diastolic blood pressure ≥about 90 mmHg, a mean arterial blood pressure ≥about 100 mmHg, a left ventricular end-diastolic pressure ≥about 14 mmHg, or a combination of these blood pressure readouts. In one embodiment, the blood pressure comprises an arterial systolic blood pressure ≥about 150 mmHg, an arterial diastolic blood pressure ≥about 110 mmHg, a mean arterial blood pressure ≥about 130 mmHg, a left ventricular end-diastolic pressure ≥about 14 mmHg, or a combination of these blood pressure readouts. In one embodiment, the blood pressure further comprises elevated exercise intolerance, and/or LV diastolic dysfunction (elevated E/e′). In one embodiment, the animal chow is provided daily as a total dry weight of about 900 grams. In one embodiment, the method further comprises providing water ad libitum to the animal. In one embodiment, the water comprises about 40 mg salt per 1 liter. In one embodiment, the animal chow and water are administered for at least 20 weeks. In one embodiment, the animal is a swine laboratory animal. In one embodiment, the swine animal is a Gottingen Minipig®.

An aspect of the invention is directed to a method for inducing diabetes in a laboratory animal. In one embodiment, the method comprises administering the composition of any one of the animal HFpEF compositions described herein to a swine laboratory animal. In one embodiment, the hypertensive agent comprises 11-deoxycorticosterone acetate (DOCA), N(gamma)-nitro-L-arginine methyl ester (L-NAME), or Angiotensin II (ANG II). In one embodiment, the hypertensive agent comprises a pellet or continuous infusion. In one embodiment, the animal chow comprises about a 50%-50% (wt/wt) proportion of standard diet and custom diet, wherein the standard diet comprises about 33% carbohydrate, about 3.9% fat, about 16.1% protein, about 0% cholesterol, about 0.4% salt, about 2.3 kcal/g energy density, or a combination of the standard diet components described herein; and the custom diet comprises about 39.8% carbohydrate, about 40% to about 44.1% fat, about 16.2% protein, about 2% cholesterol, about 0.5% to about 0.7% sodium cholate, about 4.01 kcal/g energy density, or a combination of the custom diet components described herein. In one embodiment, the custom diet further comprises about 4% salt. In one embodiment, the custom diet further comprises carbohydrates that comprise about 17.8% fructose. In one embodiment, DOCA is formulated in a dose of about 25 mg/kg to about 100 mg/kg. In one embodiment, the DOCA pellet is administered subcutaneously or orally. In one embodiment, DOCA, L-NAME, or ANG II is administered subcutaneously, intravenously, transdermal, or orally. In one embodiment, the administering occurs from 30 days to 90 days to induce and maintain high blood pressure. In one embodiment, the blood pressure comprises an arterial systolic blood pressure ≥about 130 mmHg, an arterial diastolic blood pressure ≥about 90 mmHg, a mean arterial blood pressure ≥about 100 mmHg, a left ventricular end-diastolic pressure ≥about 14 mmHg, or a combination of these blood pressure readouts. In one embodiment, the blood pressure comprises an arterial systolic blood pressure ≥about 150 mmHg, an arterial diastolic blood pressure ≥about 110 mmHg, a mean arterial blood pressure ≥about 130 mmHg, a left ventricular end-diastolic pressure ≥about 14 mmHg, or a combination of these blood pressure readouts. In one embodiment, the blood pressure further comprises elevated exercise intolerance, and/or LV diastolic dysfunction (elevated E/e′). In one embodiment, the animal chow is provided daily as a total dry weight of about 900 grams. In one embodiment, the method further comprises providing water ad libitum to the animal. In one embodiment, the water comprises about 40 mg salt per 1 liter. In one embodiment, the animal chow and water are administered for at least 20 weeks. In one embodiment, the animal is a swine laboratory animal. In one embodiment, the swine animal is a Gottingen Minipig®.

An aspect of the invention is directed to a method for inducing metabolic syndrome in a laboratory animal. In one embodiment, the method comprises administering the composition of any one of the animal HFpEF compositions described herein to a swine laboratory animal. In one embodiment, the hypertensive agent comprises 11-deoxycorticosterone acetate (DOCA), N(gamma)-nitro-L-arginine methyl ester (L-NAME), or Angiotensin II (ANG II). In one embodiment, the hypertensive agent comprises a pellet or continuous infusion. In one embodiment, the animal chow comprises about a 50%-50% (wt/wt) proportion of standard diet and custom diet, wherein the standard diet comprises about 33% carbohydrate, about 3.9% fat, about 16.1% protein, about 0% cholesterol, about 0.4% salt, about 2.3 kcal/g energy density, or a combination of the standard diet components described herein; and the custom diet comprises about 39.8% carbohydrate, about 40% to about 44.1% fat, 1 about 6.2% protein, about 2% cholesterol, about 0.5% to about 0.7% sodium cholate, about 4.01 kcal/g energy density, or a combination of the custom diet components described herein. In one embodiment, the custom diet further comprises about 4% salt. In one embodiment, the custom diet further comprises carbohydrates that comprise about 17.8% fructose. In one embodiment, DOCA is formulated in a dose of about 25 mg/kg to about 100 mg/kg. In one embodiment, the DOCA pellet is administered subcutaneously or orally. In one embodiment, DOCA, L-NAME, or ANG II is administered subcutaneously, intravenously, transdermal, or orally. In one embodiment, the administering occurs from 30 days to 90 days to induce and maintain high blood pressure. In one embodiment, the blood pressure comprises an arterial systolic blood pressure ≥about 130 mmHg, an arterial diastolic blood pressure ≥about 90 mmHg, a mean arterial blood pressure ≥about 100 mmHg, a left ventricular end-diastolic pressure ≥14 mmHg, or a combination of these blood pressure readouts. In one embodiment, the blood pressure comprises an arterial systolic blood pressure ≥about 150 mmHg, an arterial diastolic blood pressure ≥about 110 mmHg, a mean arterial blood pressure ≥about 130 mmHg, a left ventricular end-diastolic pressure ≥about 14 mmHg, or a combination of these blood pressure readouts. In one embodiment, the blood pressure further comprises elevated exercise intolerance, and/or LV diastolic dysfunction (elevated E/e′). In one embodiment, the animal chow is provided daily as a total dry weight of about 900 grams. In one embodiment, the method further comprises providing water ad libitum to the animal. In one embodiment, the water comprises about 40 mg salt per 1 liter. In one embodiment, the animal chow and water are administered for at least 20 weeks. In one embodiment, the animal is a swine laboratory animal. In one embodiment, the swine animal is a Gottingen Minipig®.

An aspect of the invention is directed to a method of treating a swine to cause it to have heart failure with preserved ejection (HFpEF) whereby the swine can be used as an animal model. In one embodiment, the method comprises administering to a swine an animal HFpEF composition; inducing high blood pressure above an arterial systolic blood pressure of about 150 mm HG in the swine by 30 days; and maintaining high blood pressure above an arterial systolic blood pressure of about 150 mm HG in the swine for at least 30 days. In one embodiment, the HFpEF composition comprises a hypertensive agent formulated in a dose of about 5 mg/kg to about 100 mg/kg, wherein the hypertensive agent comprises 11-deoxycorticosterone acetate (DOCA), N(gamma)-nitro-L-arginine methyl ester (L-NAME), or Angiotensin II (ANG II), and an animal chow comprising a 50%-50% (wt/wt) proportion of standard diet and custom diet, wherein the standard diet comprises about 33% carbohydrate, about 3.9% fat, about 16.1% protein, about 0% cholesterol, about 0.4% salt, about 2.3 kcal/g energy density, or a combination thereof, wherein the custom diet comprises about 39.8% carbohydrate, about 40% to about 44.1% fat, about 16.2% protein, about 2% cholesterol, about 0.5% to about 0.7% sodium cholate, about 4.01 kcal/g energy density, or a combination thereof. In one embodiment, the method further comprises increasing circulating LDL cholesterol levels above 150 mg/DL for 30 days. In one embodiment, DOCA is formulated in a dose of about 25 mg/kg to about 100 mg/kg. In one embodiment, the DOCA pellet is administered subcutaneously or orally. In one embodiment, DOCA, L-NAME, or ANG II is administered subcutaneously, intravenously, transdermal, or orally. In one embodiment, the custom diet further comprises 4% salt. In one embodiment, the custom diet further comprises carbohydrates that comprise 17.8% fructose. In one embodiment, the blood pressure comprises an arterial systolic blood pressure ≥about 130 mmHg, an arterial diastolic blood pressure ≥about 90 mmHg, a mean arterial blood pressure ≥about 100 mmHg, a left ventricular end-diastolic pressure ≥about 14 mmHg, or a combination of these blood pressure readouts. In one embodiment, the blood pressure comprises an arterial systolic blood pressure ≥about 150 mmHg, an arterial diastolic blood pressure ≥about 110 mmHg, a mean arterial blood pressure ≥about 130 mmHg, a left ventricular end-diastolic pressure ≥about 14 mmHg, or a combination of these blood pressure readouts. In one embodiment, the animal chow is provided daily as a total dry weight of about 900 grams. In one embodiment, the method further comprises providing water ad libitum to the animal. In one embodiment, the water comprises about 40 mg salt per 1 liter. In one embodiment, the animal chow and water are administered for at least 20 weeks. In one embodiment, the animal is a swine laboratory animal. In one embodiment, the swine animal is a Gottingen Minipig®.

An aspect of the invention is directed to a heart failure with preserved ejection (HFpEF) animal model. In embodiments, the animal model is prepared according to the methods described herein. In one embodiment, the animal is a swine laboratory animal. In one embodiment, the swine animal is a Gottingen Minipig®.

An aspect of the invention is directed to kits for heart failure with preserved ejection (HFpEF) animal model. In one embodiment, the kit comprises an animal HFpEF composition, a Gottingen Minipig® and can further comprise instructions for use. In one embodiment, the animal HFpEF composition comprises a hypertensive agent formulated in a dose of about 5 mg/kg to about 100 mg/kg, and an animal chow. In one embodiment, the hypertensive agent comprises 11-deoxycorticosterone acetate (DOCA), N(gamma)-nitro-L-arginine methyl ester (L-NAME), or Angiotensin II (ANG II). In one embodiment, the hypertensive agent comprises a pellet comprising DOCA formulated in a dose of about 25 mg/kg to about 100 mg/kg. In one embodiment, the animal chow comprises about a 50%-50% (wt/wt) proportion of standard diet and custom diet, wherein the standard diet comprises about 33% carbohydrate, about 3.9% fat, about 16.1% protein, about 0% cholesterol, about 0.4% salt, about 2.3 kcal/g energy density, or a combination of the standard diet components described herein; and the custom diet comprises about 39.8% carbohydrate, about 40% to about 44.1% fat, about 16.2% protein, about 2% cholesterol, about 0.5% to about 0.7% sodium cholate, about 4.01 kcal/g energy density, or a combination of the custom diet components described herein.

### BRIEF DESCRIPTION OF THE FIGURES

FIG. 1 is a schematic of a components of the Standard Diet under one embodiment.

FIG. 2 is a schematic of a components of the Custom Diet under one embodiment.

FIG. 3 is a schematic of a Gottingen Minipig® HFpEF experimental protocol using DOCA.

FIG. 4 is a schematic of a Gottingen Minipig® HFpEF experimental protocol using L-NAME.

FIG. 5 is a schematic of a Gottingen Minipig® HFpEF experimental protocol using Angiotensin.

FIG. 6 is a schematic of an experimental protocol using DOCA. In this experimental protocol six Gottingen Minipigs® were utilized, three of which were fed a standard diet and the remaining three were fed a custom diet plus DOCA. Figure Legend: \hat{ }=Body Weight & Blood Pressure Cuff; *=Peripheral Blood draw; †=Transthoracic Echo; Δ=Invasive Hemodynamics; #=Tissue Harvest for Molecular and Histopathology Analysis.

FIG. 7 is a graph showing survival percentages.

FIG. 8 is a graph showing body weights.

FIG. 9 is a group of graphs showing circulating lipid profiles.

FIG. 10 is a group of graphs showing left ventricular volumes and functions.

FIG. 11 is a group of graphs showing left ventricular internal dimensions.

FIG. 12 is a group of graphs showing left ventricular wall thickness.

FIG. 13 is a graph showing relative wall thickness.

FIG. 14 is a group of graphs showing flow velocity and tissue doppler assessment of diastolic function.

FIG. 15 is a group of graphs showing 8 week left ventricular pressures.

FIG. 16 is a group of graphs showing left atrial structure.

FIG. 17 is a group of graphs showing 8 week pre- & post-capillary pulmonary.

FIG. 18 is group of pictures showing left ventricular hypertrophy and aortic wall thickening.

FIG. 19 is a group of images showing atherosclerotic lesions in the aorta.

FIG. 20 is a group of images showing atherosclerotic lesions in the aorta.

FIG. 21 is a group is histology images of the liver 8 weeks post Western Diet+DOCA.

FIG. 22 is a group of histology images showing kidney cortex 8 weeks post Western Diet+DOCA.

FIG. 23 is a schematic of an experimental protocol using DOCA. (Control (n=3); HFpEF Diet+DOCA (WD+DOCA) (n=5). Figure Legend: *=Body Weight, Peripheral Blood Draw (Lipid Profile (0, 4, 12, and 20 weeks), Transthoracic Echo (LV Volumes, LV & LA Dimensions, PW Doppler Transmitral Inflow Velocities, Tissue Doppler Imaging of Mitral Valve Annulus); ▴=Invasive Hemodynamics (Arterial Blood Pressure, Left Ventricular Pressures, Pulmonary Pressures); #=DOCA Pellet Implantation (50 mg/kg, 60-Day release); ♦=Intravenous Glucose Tolerance Test (measure blood glucose, insulin: −5 min., 0, 2.5 min., 5 min., 10 min., then every 10 min. for a total of 60 min.); ⊙=Tissue Harvest for Molecular and Histopathology Analysis (Masson's Trichrome Staining).

FIG. 24 is a graph of body weight.

FIG. 25 is a group of graphs showing results of intravenous glucose tolerance tests at 20 weeks.

FIG. 26 is a pair of graphs showing circulating lipid profiles.

FIG. 27 is a group of graphs showing arterial blood pressure.

FIG. 28 is a graph showing left ventricular function.

FIG. 29 is a group of graphs showing diastolic function. The graphs on the left provide tissue doppler at 20 weeks.

FIG. 30 is a pair of graphs showing results of hypertrophic remodeling.

FIG. 31 is a pair of graphs showing left ventricular blood pressure.

FIG. 32 is a group of graphs showing left arterial structure and function.

FIG. 33 is a group of graphs showing pulmonary pressures.

FIG. 34 is a graph showing pulmonary artery wedge pressure.

FIG. 35 is a group of graphs showing coronary artery (LAD) vascular reactivity.

FIG. 36 is a group of graphs showing coronary artery (LCX) vasculary reactivity.

FIG. 37 is a graph and histology images showing LV fibrosis staining and quantification.

FIG. 38 is a pair of graphs and histology images showing renal fibrosis staining and quantification.

FIG. 39 is an experimental protocol of a new “multi-hit” minipig model of HFpEF. Figure Legend: †=Body Weight, 2D Echocardiography; *=Circulating Lipid Profile; ⋄=Invasive Hemodynamics (Arterial Blood Pressures, Left Ventricular Pressures, Pulmonary Pressures); Δ=Intravenous Glucose Tolerance Test; ∘=Ex vivo Vascular Reactivity and Tissue Fibrosis. Female Gottingen minipigs were subjected to 20 weeks of excess mineralocorticoid exposure and dietary insults to drive development of prominent comorbidities associated with HFpEF: obesity, hypercholesterolemia, pre-diabetes, pulmonary and systemic arterial hypertension. Adult female Gottingen miniswine were randomized to the Control group (n=3) and fed a standard diet or to the HFpEF group (n=5) and received subcutaneous implantation of deoxycorticosterone acetate (DOCA) pellets (50 mg/kg, 60-day release) and fed a custom Western diet high in cholesterol (1%), fat (21%), fructose (8.9%) and salt (2%). Body weights, circulating lipid levels and transthoracic echocardiographic assessment of cardiac function and structure were evaluated at baseline and 4, 8, 12, 16 and 20 weeks. Intravenous glucose tolerance testing and invasive hemodynamics were performed at baseline and 20 weeks. At 20 weeks, hearts and kidneys were collected, coronary arteries isolated for ex vivo vascular reactivity experiments and histological tissue fibrosis quantified in left ventricular myocardium and renal cortex samples.

FIG. 40 is a group of graphs showing the development of obesity, dyslipidemia, glucose intolerance and risk biomarkers of cardiovascular disease. Body weights (Panel A) were obtained at baseline and every 4 weeks throughout the 20 week study. Lipid profiling was performed at baseline, 4, 12 and 20 weeks. Total cholesterol (TC) (Panel B), low-density lipoprotein (LDL) (Panel C) and ratio of TC to high-density lipoprotein (HDL) (Panel D) were measured. At 20 weeks, conscious intravenous (IV) glucose tolerance testing was performed in overnight fasted minipigs. Glucose levels (Panel E) were measured from timed blood samples obtained over 60 min following IV glucose administration. The natural log (Ln) of glucose (Panel F) determined 5-30 min following IV glucose administration was calculated. Insulin levels (Panel G) were measured from timed blood samples obtained over 60 min following IV glucose administration. Circulating plasma 8-isoprostane (Panel H) levels were measured at baseline, 4, 12 and 20 weeks. Serum endothelin-1 levels were measured at baseline, 4, 12 and 20 weeks (Panel I). Values are mean±SEM; p=NS, not significant; *p<0.05; **p<0.01; for significantly different versus control. Numbers in circles represent the number of animals analyzed.

FIG. 41 is a group of graphs showing DOCA-salt hypertension and preserved left ventricular ejection fraction. Baseline and 20 week arterial blood pressures were acquired and systolic blood pressure (SBP) (Panel A), diastolic blood pressure (DBP) (Panel B) and mean arterial pressure (MAP) (Panel C) recorded. Echocardiography was performed at baseline and every 4 weeks throughout the 20 week study. Left ventricular (LV) end-systolic volume (ESV) (Panel D) and end-diastolic volume (EDV) (Panel E) were measured. LV ejection fraction (LVEF) (Panel F) was calculated from LVESV and LVEDV. Values are mean±SEM; p=NS, not significant. Numbers in circles represent the number of animals analyzed.

FIG. 42 is a group of graphs showing increased E/e′ ratio indicative of left ventricular diastolic dysfunction. Indicators of cardiac filling pressures were evaluated non-invasively at baseline and every 4 weeks throughout the 20 week study, transmitral inflow velocity was measured by pulsed-wave Doppler echocardiography and mitral annular velocity was measured by pulsed-wave tissue doppler imaging. Representative trace recordings (Panel A) of mitral inflow (E/A) and tissue Doppler medial and lateral mitral annular velocities (e′) at 20 weeks acquired from control and HFpEF minipigs. Peak early (E) and late atrial (A) diastolic transmitral flow velocity along with early (e′) and late atrial (a′) diastolic mitral annular velocity are noted. Early to late diastolic transmitral flow velocity (E/A) ratios (Panel B) and the E to early diastolic mitral medial (Panel C) and lateral (Panel D) annular tissue velocity (E/e′) ratios were calculated. Values are mean±SEM; p=NS, not significant; *p<0.05; **p<0.01; for significantly different versus control. Numbers in circles represent the number of animals analyzed.

FIG. 43 is a group of graphs and images showing pathophysiological concentric hypertrophy of the left ventricle. Representative transthoracic echocardiographic subcostal 2D B-mode images of the left ventricle (Panel A) acquired at baseline and 20 weeks from control and HFpEF minipigs. Left ventricular chamber internal diameter (LVID) (Panel B) and thickness of interventricular septum (IVS) (Panel C) and LV posterior wall (LVPW) (Panel D) were measured from images during end-diastole (d). LV relative wall thickness (Panel E) and LV mass (Panel F) were derived using the end-diastolic linear measurements of the LVID, IVS, and LVPW measurements. Representative photomicrographs of gross mid-papillary cross-sections from a control and HFpEF heart (Panel G). Scale bar=2 cm. Values are mean±SEM; p=NS, not significant; *p<0.05; **p<0.01; for significantly different versus control. Numbers in circles represent the number of animals analyzed.

FIG. 44 is a group of graphs showing elevated left ventricular filling pressures promote left atrial dysfunction leading to combined precapillary and postcapillary pulmonary hypertension. Invasive left ventricular (LV) hemodynamic measurements were acquired at baseline and 20 weeks from control and HFpEF minipigs. LV end-diastolic pressure (LVEDP) (Panel A) was calculated. B-type natriuretic peptide (Panel B) and atrial natriuretic peptide (Panel C) mRNA expression of left ventricular tissue. Left atrial (LA) area at end-diastole (ED) (Panel D) and fractional area change (LAFAC) (Panel E) were measured from subcostal 2D B-mode echocardiographic images acquired at baseline and every 4 weeks during the 20 week study from control and HFpEF minipigs. Pulmonary artery systolic (PASP) (Panel F) and pulmonary capillary wedge pressure (PCWP) (Panel G) and central venous pressure (CVP) (Panel H) were measured at baseline and 20 weeks in control and HFpEF minipigs. The relationship of body weight to PCWP at baseline and 20 weeks was plotted (Panel I). Values are mean±SEM; p=NS, not significant; **p<0.01; for significantly different versus control. Numbers in circles represent the number of animals analyzed.

FIG. 45 is a group of graphs showing impaired nitric oxide-mediated coronary artery relaxation. Left anterior descending (LAD) (Panels A, C, E) and left circumflex (LCX) (Panels B, D, F) coronary arteries were isolated at 20 weeks from control and HFpEF minipig hearts for isometric tension experiments. Coronary arteries were precontracted with PGF2a and relaxation response curves to endothelial-dependent bradykinin (Panels A, B) and substance P (Panels C, D), and endothelial-independent relaxation curves to sodium nitroprusside (SNP) (Panels E, F) were generated and half maximal effective concentrations (EC50) calculated. Values are mean±SEM; p=NS, not significant; *p<0.05; **p<0.01; for significantly different versus control. Numbers in circles represent the number of animals analyzed.

FIG. 46 is a group of histology images and graphs showing multi-organ histopathology and fibrosis. At 20 weeks, hearts, lungs, liver and kidneys were collected from control and HFpEF minipigs. Tissue samples were processed for histological Masson's trichrome staining, micrographs were acquired and amount of fibrosis quantified. Representative low (4X) and high (20X) magnification images of left ventricular (LV) myocardial tissue (Panel A) lung tissue (Panel C), liver tissue (Panel E) and renal cortical tissue (Panel G) samples from control and HFpEF minipigs are shown. Percentage of fibrosis area of Masson's trichrome-stained LV myocardial (Panel B), lung (Panel D), liver (Panel F) and renal cortical (Panel D) tissue sections were quantified. Scale bars: 4×, 200 μM; 20×, 50 μM. Values are mean±SEM. Numbers in circles represent the number of animals analyzed.

FIG. 47 shows a schematic of an experimental protocol using (L-NAME).

FIG. 48 shows a schematic of an experimental protocol using Angiotensin II infusion.

### DETAILED DESCRIPTION OF THE INVENTION

### Abbreviations and Definitions

Detailed descriptions of one or more embodiments are provided herein. It is to be understood, however, that the invention can be embodied in various forms. Therefore, specific details disclosed herein are not to be interpreted as limiting, but rather as a basis for the claims and as a representative basis for teaching one skilled in the art to employ the invention in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be non-limiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

In embodiments, the at least a portion of the HFpEF compositions described herein can be provided as daily feed ration for laboratory animals. For example, laboratory animals can include, mice, rats, donkeys, swine (pigs), dogs, cats, rabbits, horses, sheep, goats, monkeys, and non-human primates. For example, the laboratory animal is a Gottingen Minipig®. As used herein, the term “standard diet,” “control diet,” or “standard control diet” can refer to a normal, calorically balanced maintenance diet that is not designed to induce hypertension or dyslipidemia. In an embodiment, the standard diet comprises a balanced maintenance diet employed for optimum health of the animal. For example, a standard diet will not result in hypertension, obesity, metabolic syndrome, and/or diabetes mellitus in animals and animal models that do not have a predisposition for any of the foregoing. As used herein, the term “standard diet” can be used interchangeably with the term “control diet” and “standard control diet.” As used herein, the term “custom diet” can refer to a diet that can result in an outcome by the consumer of said diet. In embodiments, a Western Diet in combination with a Standard Diet can refer to an HFpEF diet. In an embodiment, a percentage of Western diet can be added to a percentage of a standard diet to create an animal chow. In certain embodiments, a portion of Western Diet can be combined with a portion of a standard diet to create an animal chow. An animal chow can comprise a portion of Western Diet that is about equal to a portion of the standard diet. modified custom. In embodiments, animal chow comprises about a 50/50 mixture (by weight) of Western Diet and standard control diet. The term “animal chow” and “HFpEF diet” can be used interchangeably. In some embodiments, the term “Western Diet” and the term “custom diet” can be used interchangeably. In embodiments, a Western Diet or custom diet can refer to a diet high in fat, fructose, salt, or a combination thereof. In embodiments, the standard diet comprises about 33% carbohydrate, about 3.9% fat, about 16.1% protein, about 0% cholesterol, about 0.4% salt, about 2.3 kcal/g energy density, or a combination thereof. In embodiments, the Western Diet or custom diet comprises about 39.8% carbohydrate, about 40% to about 44.1% fat, about 16.2% protein, about 2% cholesterol, about 0.5% to about 0.7% sodium cholate, about 4.01 kcal/g energy density, or a combination thereof. In embodiments, the standard diet comprises about 33% carbohydrate, about 3.9% fat, about 16% protein, about 0% cholesterol, about 0.4% salt, about 2.3 kcal/g energy density, or a combination thereof. In embodiments, the custom diet comprises about 39.9% carbohydrate, about 40% to about 44.1% fat, about 16.2% protein, about 2% cholesterol, about 0.5% to about 0.7% sodium cholate, about 4.01 kcal/g energy density, or a combination thereof.

In embodiments, the animal chow herein can comprise a high fat content. The fat included in the animal chow may come from more than one fat source. In some embodiments, the animal chow is in the form of feed particles. In some embodiments, a combination of at least two, at least three, at least four, or at least 5 fats are used. Feed particles may be made by methods known in the art.

As used herein, the term “hypertensive agent” can refer to any compound that when administered to a subject increases blood pressure. In embodiments, the hypertensive agent comprises 11-deoxycorticosterone acetate (DOCA), N(gamma)-nitro-L-arginine methyl ester (L-NAME), or Angiotensin II (ANG II). As used herein, the term “blood pressure” refers to the pressure of the blood within the arteries.

In embodiments, an HFpEF composition comprises animal chow and a hypertensive agent.

In one embodiment, the HFpEF composition comprises a hypertensive agent formulated in a dose of less than 5 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, and greater than 100 mg/kg.

In an embodiment, the animal chow comprises about 10%-90% wt/wt proportion of standard diet and custom diet, about 20%-80% wt/wt proportion of standard diet and custom diet, about 25%-75% wt/wt proportion of standard diet and custom diet, about 30%-70% wt/wt proportion of standard diet and custom diet, about 40%-60% wt/wt proportion of standard diet and custom diet, about 50%-50% wt/wt proportion of standard diet and custom diet, about 55%-45% wt/wt proportion of standard diet and custom diet, about 60%-40% wt/wt proportion of standard diet and custom diet, about 65%-35% wt/wt proportion of standard diet and custom diet, about 70%-30% wt/wt proportion of standard diet and custom diet, about 75%-25% wt/wt proportion of standard diet and custom diet, about 80%-20% wt/wt proportion of standard diet and custom diet, about 85%-15% wt/wt proportion of standard diet and custom diet, about 90%-10% wt/wt proportion of standard diet and custom diet, and about 95%-5% wt/wt proportion of standard diet and custom diet.

In an embodiment, the HFpEF compositions described herein can be used for administration to laboratory animals for the induction of heart failure with preserved ejection fraction (HFpEF) for the purposes of modeling the disease. In embodiments, the HFpEF model described herein recapitulates several comorbidities of HFpEF. In embodiments, comorbidities of HFpEF can comprise hypertension, old age, obesity, hypercholesterolemia, pre-diabetic phenotype, pulmonary and/or systemic arterial hypertension, diabetes mellitus, or a combination of the co-morbidities described herein.

In embodiments, the animal chow (HFpEF diet) described herein may include fat, nutritional components and other additives. For example, nutritional components can include starch, carbohydrates, and protein components. For example, other additives can include amino acids, vitamins, minerals, fatty acids, nutraceuticals, pharmaceuticals, and the like. In embodiments, additives can be added to the nutritional components or fat components of the animal chow. In embodiments, the animal chow can comprise starches such as corn, wheat, barley, oats, sorghum, tapioca, isolated dry or wet milled starch, their milled components or a combination thereof. For example, amino acids can comprise aspartic acid, glutamic acid, alanine, glycine, threonine, proline, serine, leucine, isoleucine, valine, phenylalanine, tyrosine, methionine, cystine, lysine, histidine, arginine, and tryptophan. For example, minerals can comprise calcium, phosphorus, non-phytate phosphorus, sodium, potassium, chloride, magnesium, sulfur, zinc, manganese, chloride, fluorine, cobalt, chromium, copper, iodine, iron, and selenium. For example, vitamins can comprise Vitamin A, Vitamin D3, Vitamin E, Vitamin K, Vitamin B2, Vitamin B12, thiamin, riboflavin, menadione, pantothenic acid, pyridoxine, ascorbic acid, niacin, biotin, carotene, folic acid, and choline chloride. For example, fatty acids can comprise palmitic acid, stearic acid, oleic acid, linoleic acid, arachidonic acid, linolenic acid, and omega-3 fatty acids.

In embodiments, the animal chow described herein comprise protein components. For example, protein sources can comprise soybean meal, amino acids, casein, cottonseed meal, and corn gluten meal, other oil seed meals, animal-by products, plant by-products, and microbial protein. In embodiments, the composition comprises at least one source of fiber.

In embodiments, the animal chow comprises fructose, dehulled soybean meal, wheat middlings, hydrogenated soy oil, casein, ground soybean hulls, ground corn, high fructose corn syrup-55, hydrogenated coconut oil, lard, salt, dicalcium phosphate, glucose, cholesterol, soybean oil, cane molasses, calcium carbonate, sodium cholate, potassium bicarbonate, powdered cellulose, vitamin/mineral premix (trace mineral premix, sodium selenite, pyridoxine hydrochloride, biotin, calcium pantothenate, folic acid, D-Alpha Tocopheryl Acetate, cholecalciferol, zine oxide, Vitamin B-12 supplement, Vitamin A Acetate, riboflavin supplement, sucrose, eat carotene, nicotinic acid, thiamine mononitrate, calcium iodate, copper sulfate, chromium chloride), magnesium oxide, dicalcium phosphate, menadione dimethylpyrimidinol, bisulfate, ethoxyquin.

### EXAMPLES

The following examples are given for the purpose of illustrating various embodiments of the invention and are not meant to limit the present invention in any fashion. One skilled in the art will appreciate readily that the present invention is well adapted to carry out various embodiments of the invention. Changes therein and other uses which are encompassed within the spirit of the invention as defined by the scope of the claims will occur to those skilled in the art.

Examples are provided herein to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Composition and Methods of Developing a Swine Model of Heart Failure with Preserved Ejection Fraction

Composition

1. Gottingen Minipig®

2. DOCA

3. HFpEF Diet

4. Salt

5. Water

Methods

1) The use of the Gottingen minipig (Marshall BioResources, Inc) is used to provide the proper genetic predisposition for development of a swine model of heart failure with preserved ejection fraction (HFpEF).

2) The use of 11-deoxycorticosterone acetate (DOCA) is utilized to provide proper development of this swine model of HFpEF. DOCA is administered in pellet form and is implanted subcutaneously. DOCA pellets are manufactured by Innovative Research of America, Inc. A dose range of ≥25-<100 mg/kg; for a 30-90 day release is utilized to induce and maintain high blood pressure throughout the protocol (arterial systolic blood pressure ≥130 mmHg, arterial diastolic blood pressure ≥90 mmHg and mean arterial blood pressure ≥100 mmHg). The DOCA could be delivered in alternative routes of administration.

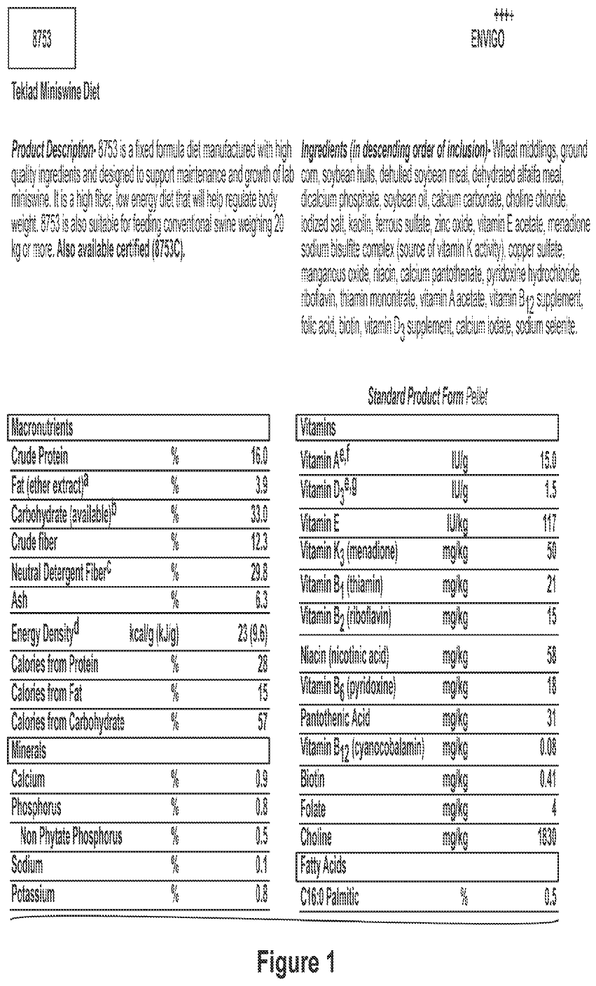

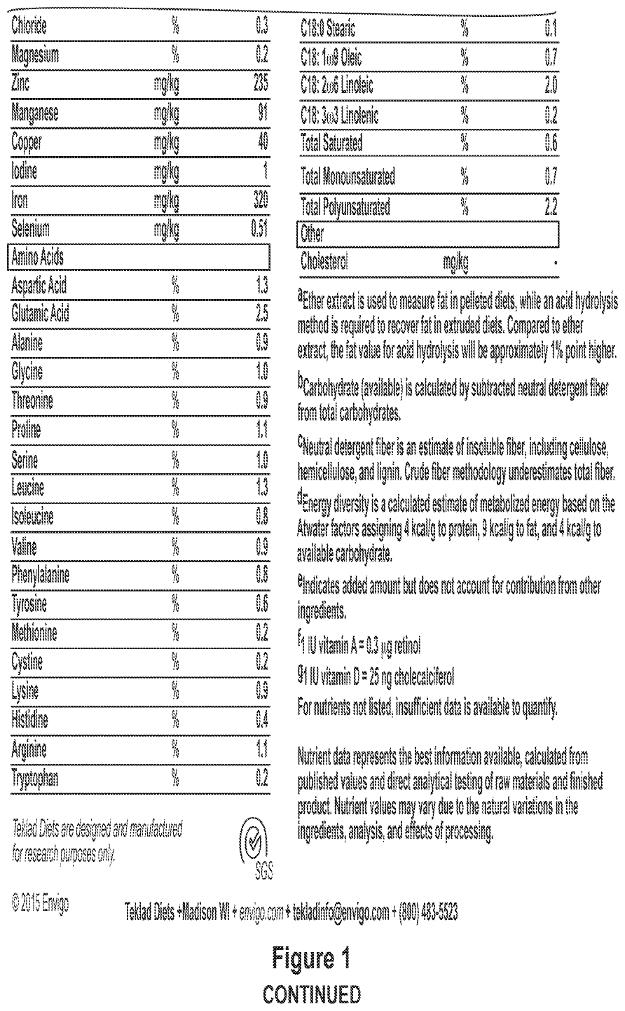

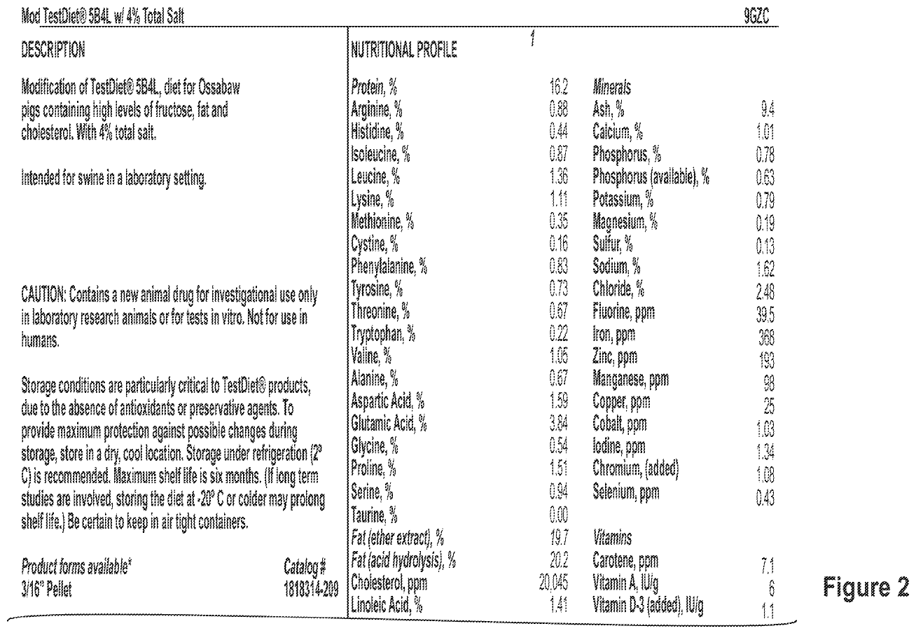

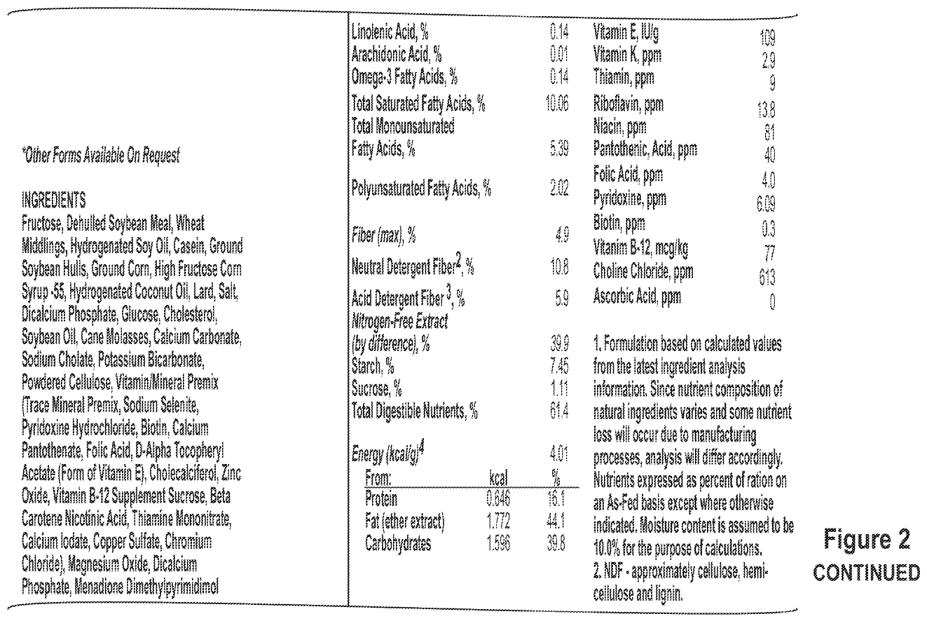

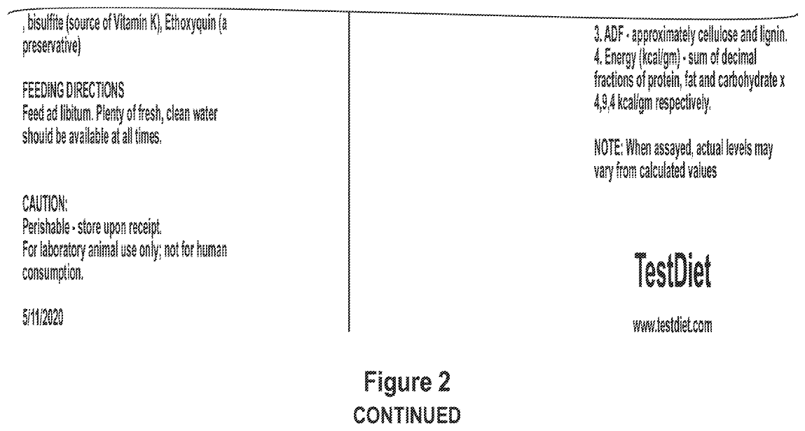

The use of a diet composed of a 50/50 (wt/wt) proportion of standard diet and custom diet is necessary to provide proper development of this swine model of HFpEF. A total dry weight of 900 grams of the newly composed 50/50 diet (termed HFpEF diet) is provided to the animals daily. The HFpEF diet is used to induce hypercholesterolemia, pre-diabetic mellitus condition, and to aid in the induction and maintenance of high blood pressure.
a. Standard diet component summary—
   i. Catalog #: 8753, Teklad Miniswine Diet, Envigo; 2.3 kcal/g⁻¹; carbohydrates, 33%; fat, 3.9%; protein, 16%; cholesterol, 0% wt/wt; 0.4% salt. Data sheet provides further composition details in FIG. 1.
b. Custom diet component summary—
   i. Catalog #: 9GZC (5B4L w/ 4% total salt), Test Diet; 4.01 kcal/g⁻¹; carbohydrates, 39.8% [fructose, 17.8%]; fat, 44.1%; protein, 16.1%; cholesterol, 2% wt/wt; 4% salt. Data sheet provides further composition details in FIG. 2.

The salt component in this instance is supplemented into the custom diet. Alternatively, the salt could be administered through the drinking water.

Access to drinking water ad libitum is necessary to induce and maintain high blood pressure.

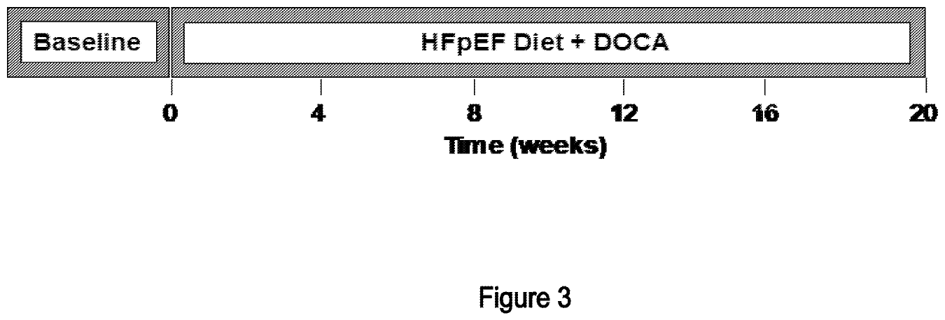

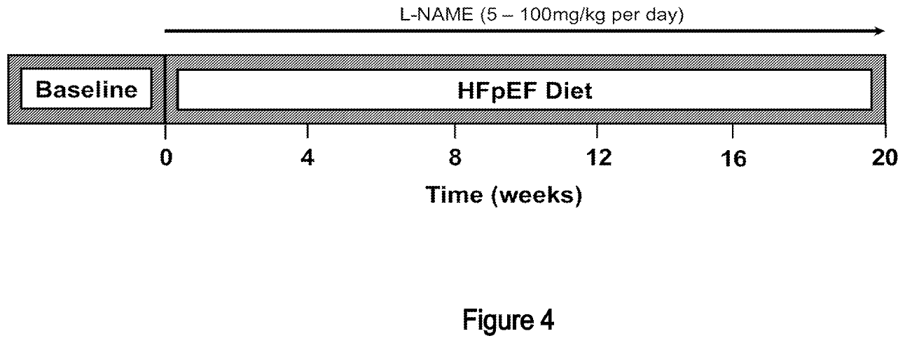

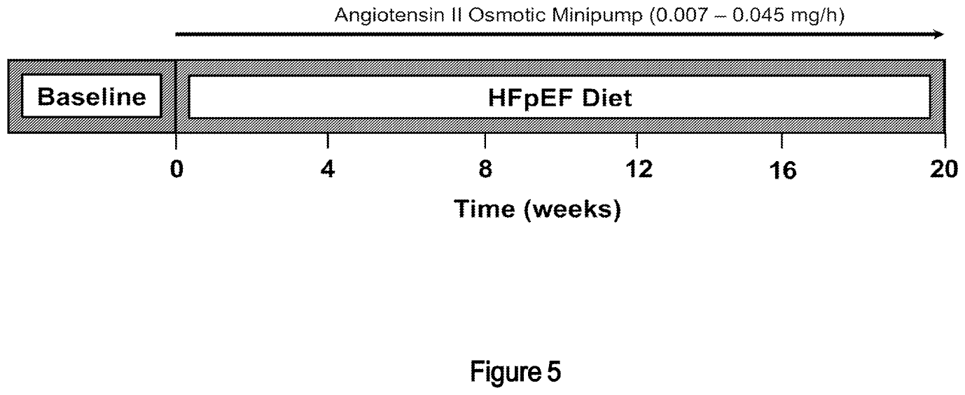

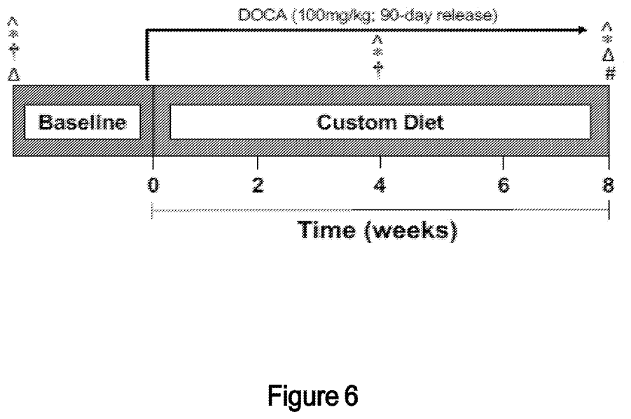

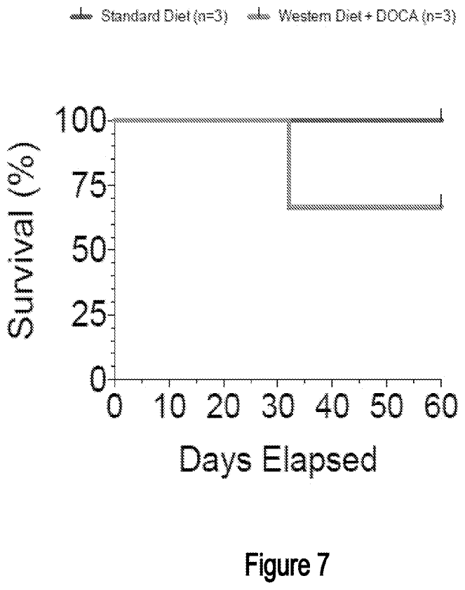

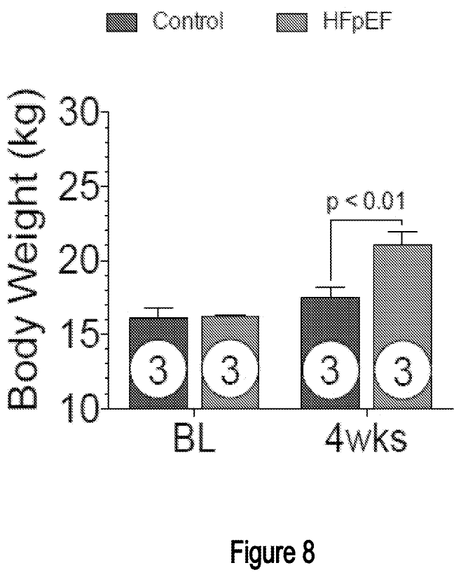

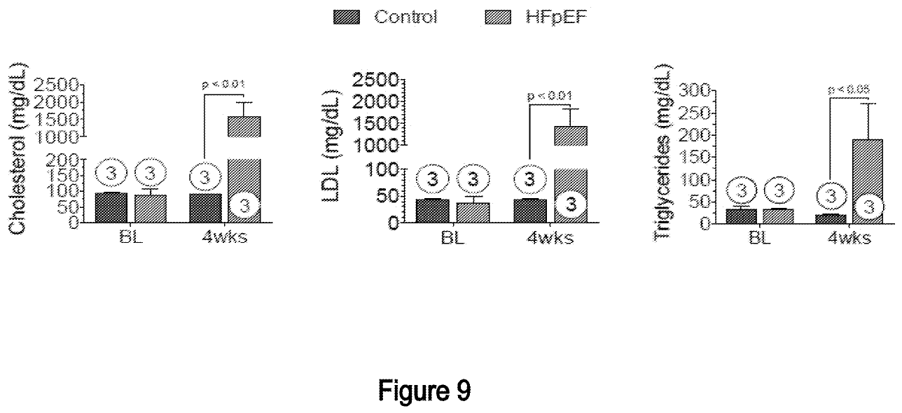

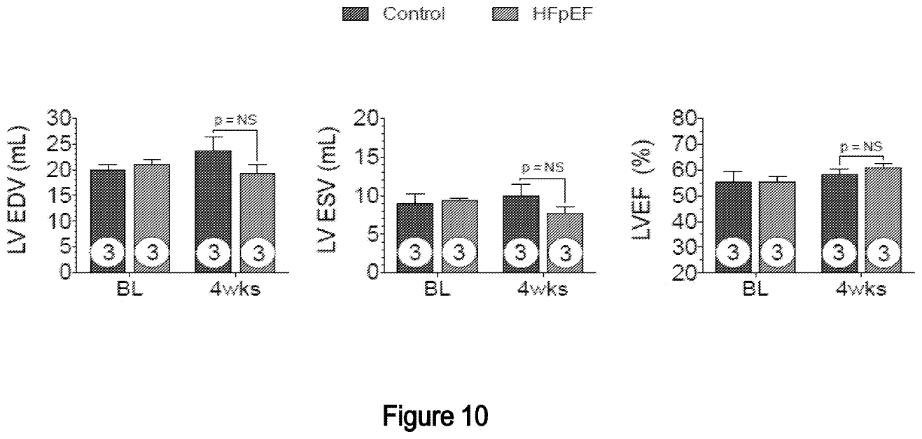

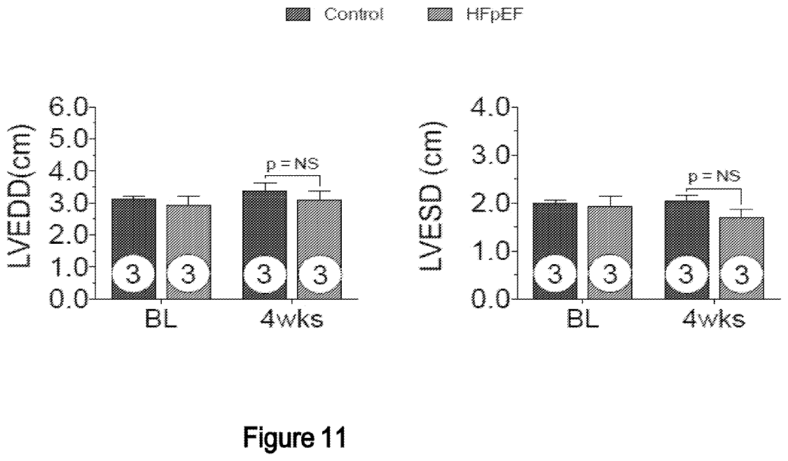

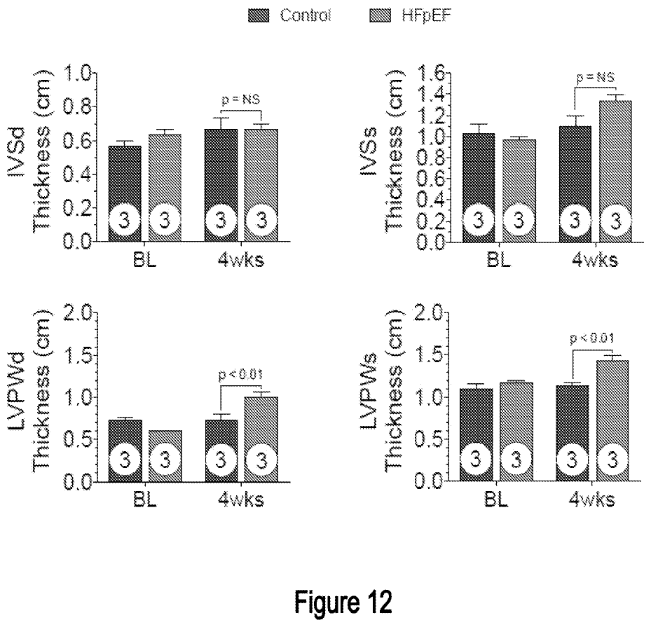

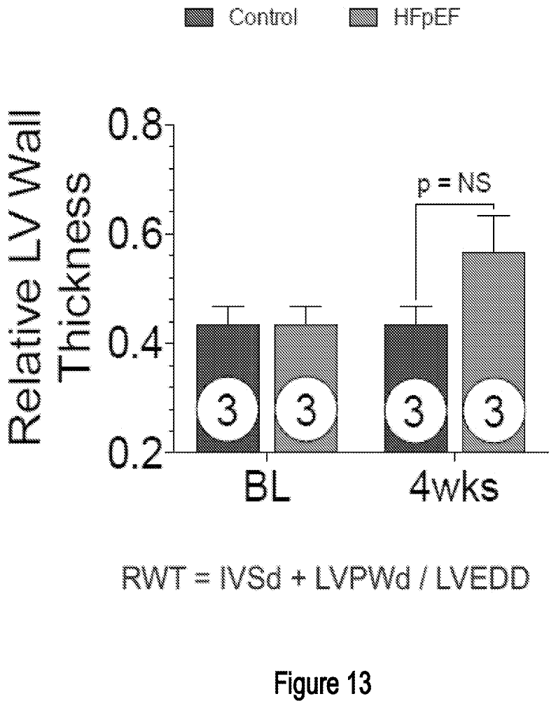

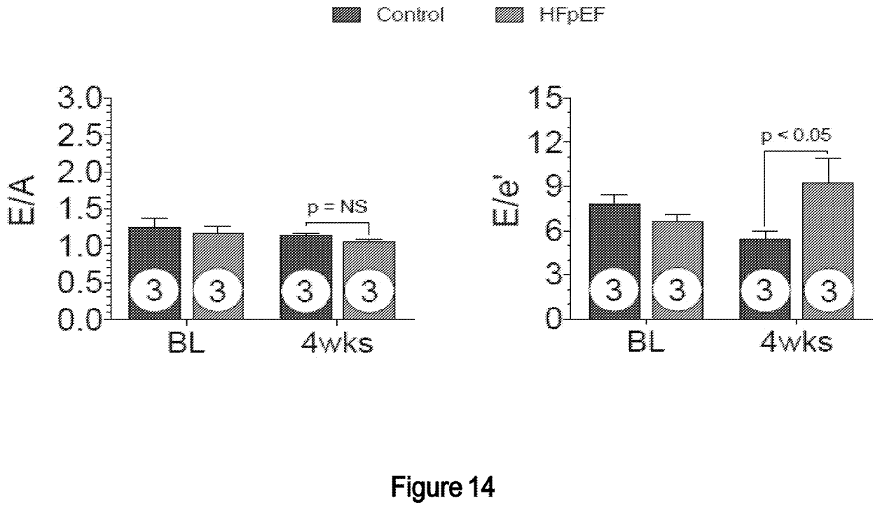

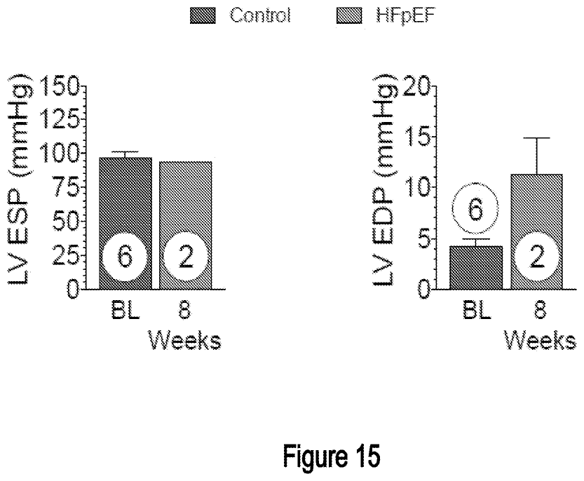

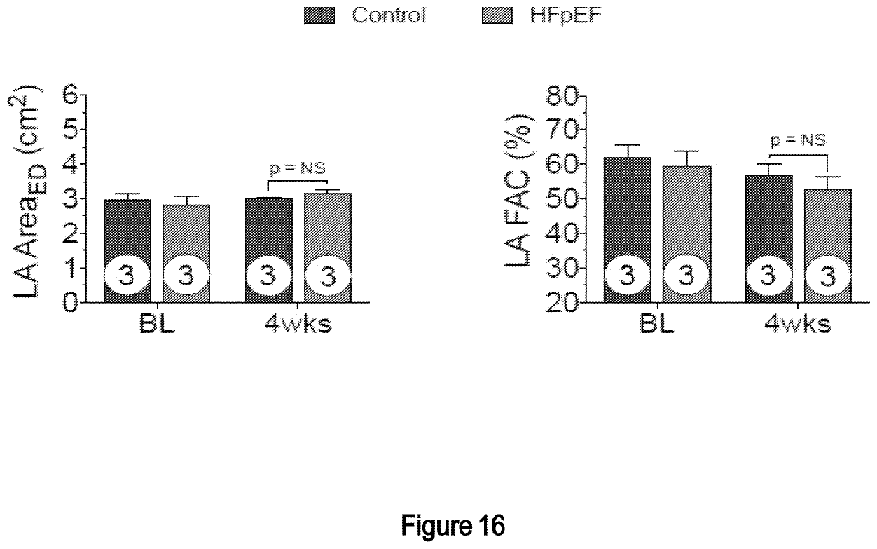

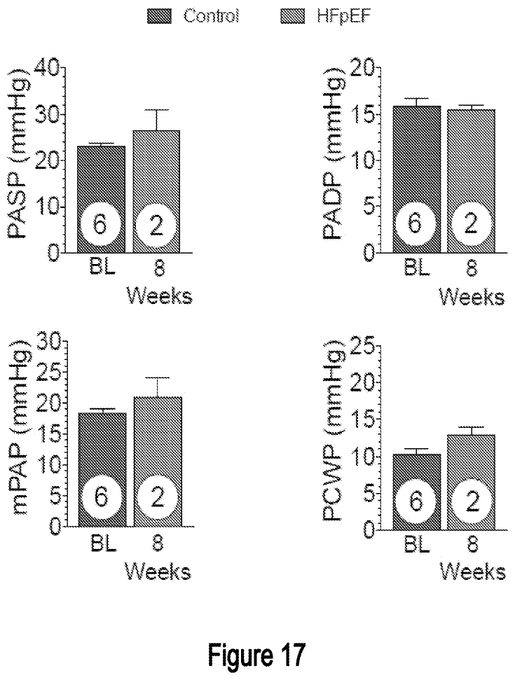

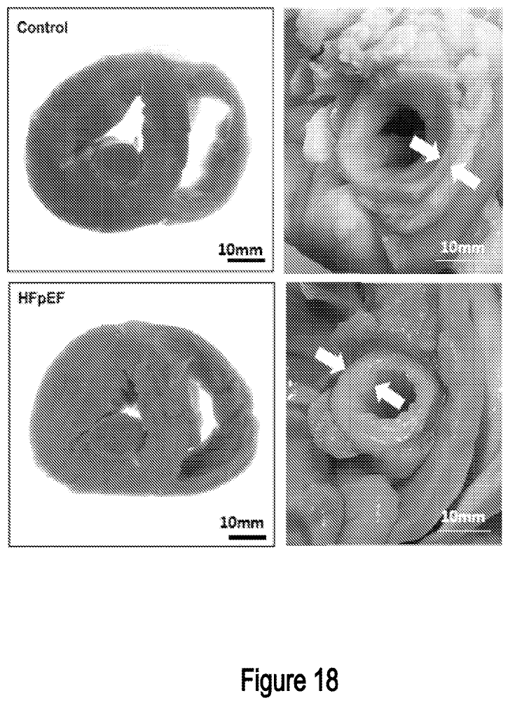

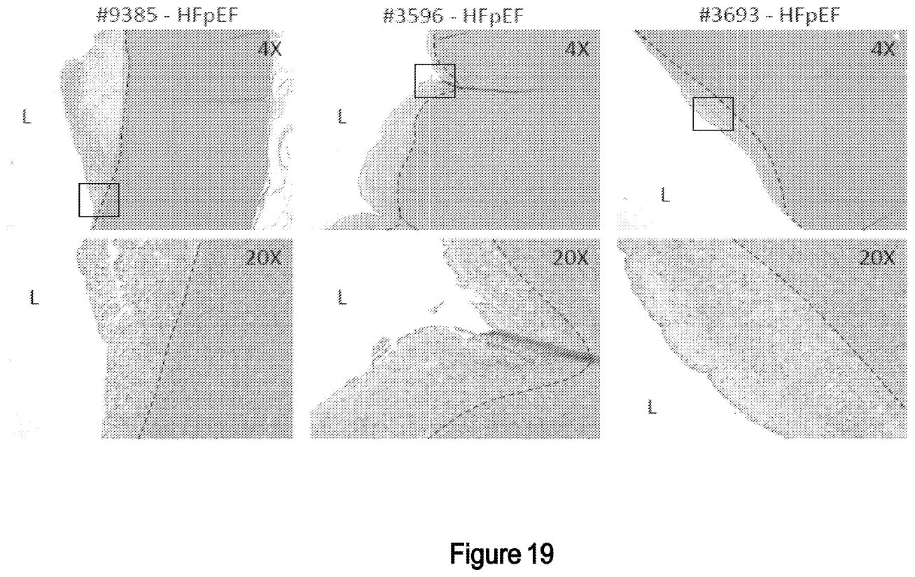

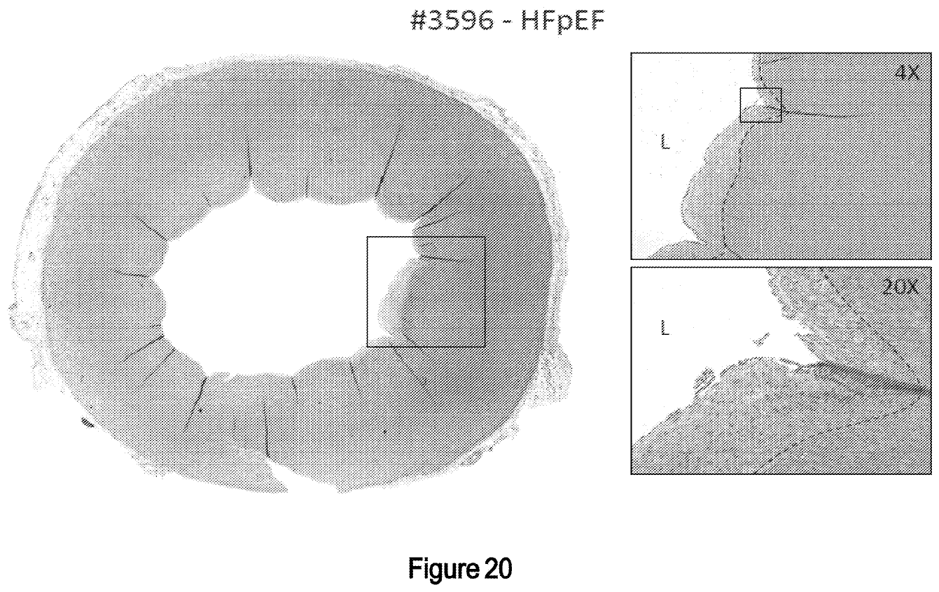

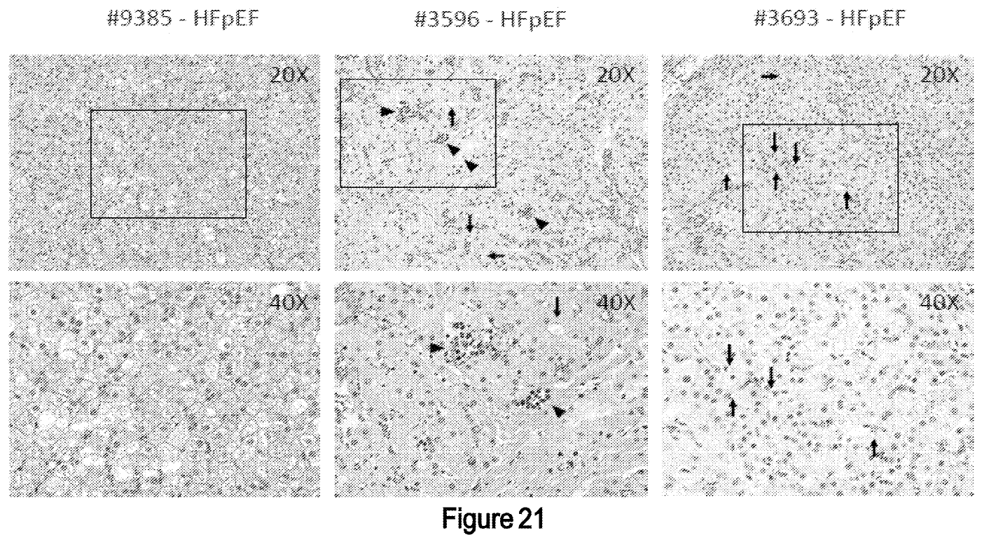

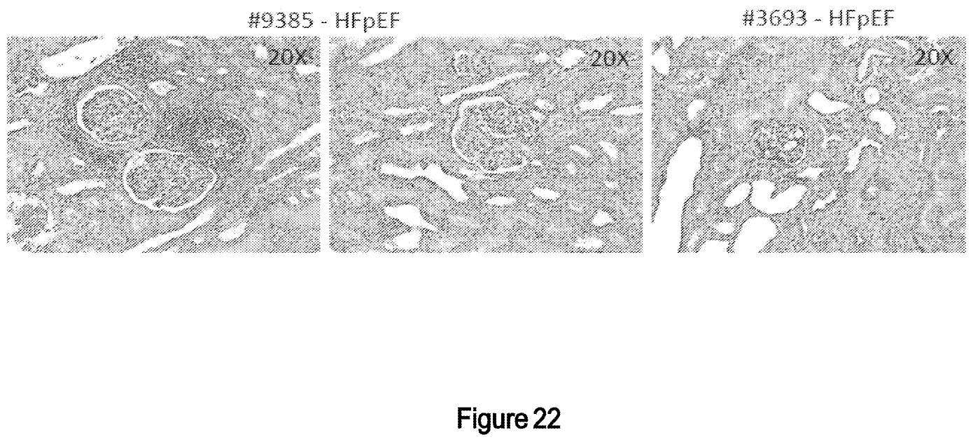

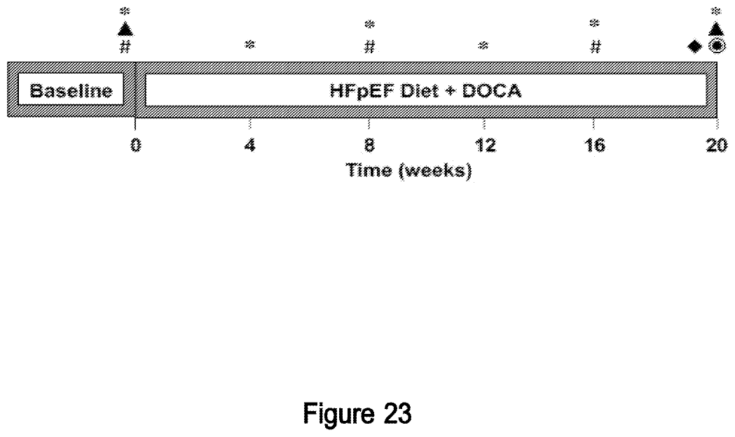

The combination of HFpEF diet, DOCA and water is maintained in the Gottingen minipig for 20 weeks (5 months) in order to develop a swine HFpEF model (FIG. 3).

### Example 2—Induction of Hypercholesterolemia and Hypertension

Hypercholesterolemia

1. Custom diet (WD)—

Ossabaw High Fat High Fructose

(TestDiet® 5B4L w/ 4% Total Salt—9GZC)
Energy (kcal/g): 4.01, Carbohydrate: 39.8%, Protein 16.1%, Fat: 44.1%
Cholesterol: 2% wt/wt
Total Salt: 4%

Hypertension (HTN)

1. Deoxycorticosterone acetate (DOCA) pellets

(Innovative Research of America SM-121) (100 mg/kg/day [90-day release])

Implanted (S.C.) behind the left ear

2. Dietary Salt (approximate 4%)

### Example 3—Induction of Hypercholesterolemia and Hypertension

Hypercholesterolemia

1. HFpEF Diet—Mixture (50/50) of Standard Dietz and Custom Diet

Standard Diet—

Mini-Pig Breeder (ENVIGO 8753)
Energy (kcal/g): 2.3, Carbohydrate: 33%, Protein 16%, Fat: 3.9%
Cholesterol: 0%
Total Salt: 0.4%

Custom Diet—

Ossabaw High Fat High Fructose

(TestDiet® 5B4L w/ 4% Total Salt—LT479)
Energy (kcal/g): 4.01, Carbohydrate: 39.8%, Protein: 16.1%, Fat: 44.1%
Cholesterol: 2% wt/wt
Total Salt: 4%

Hypertension (HTN)

1. Deoxycorticosterone acetate (DOCA) pellets

(Innovative Research of America SM-121) (50 mg/kg/day [60-day release])

Implanted (S.C.) behind the left ear

2. Dietary Salt (approximate 2.2%)

### Example 3

A large animal model that recapitulates the clinical heterogeneity of heart failure with preserved ejection fraction has been developed.
Multiple comorbidities including obesity, hypercholesterolemia, pre-diabetic phenotype, pulmonary and systemic arterial hypertension were induced in adult female Gottingen minipigs by mineralocorticoid-excess and a diet high in cholesterol, fat, fructose and salt.
Severity of each comorbidity was titrated such that marked left ventricular and left atrial diastolic dysfunction, profound left ventricular concentric remodeling, impaired coronary vasorelaxation and extensive multi-organ fibrosis was achieved over a timeframe.
This heart failure model was generated in Gottingen minipigs to allow for translational studies of pharmacological agents and for human-sized device testing in adult aged animals.
This “multi-hit’ minipig heart failure model enables investigations into the pathology of HFpEF without the confounding effects inherent with invasive surgical procedures or the administration of agents that induce hypertension and end-organ damage in an artificial manner.

### ABSTRACT

Background—Increasing prevalence of heart failure with preserved ejection fraction (HFpEF) coupled with a lack of effective therapies represents a significant unmet need in cardiovascular medicine. Likewise, a lack of preclinical animal models which recapitulate the comorbid-laden syndrome has led to the inability to tease out mechanistic insights and test new therapeutic strategies. Herein, we developed a preclinical large animal model integrating multiple co-morbid determinants of HFpEF in a miniswine breed that exhibits sensitivity to obesity, metabolic syndrome and vascular disease with overt clinical signs of heart failure.

Methods and Results—Female Gottingen miniswine were fed a standard diet or Western diet (WD) to induce metabolic syndrome coupled with 11-deoxycorticosterone acetate (DOCA)—salt induced hypertension over a 20-week period. Serial echocardiographic assessment was performed over the 20-weeks. Invasive hemodynamics were measured at baseline and 20-weeks. Gottingen miniswine treated with WD+DOCA demonstrated obesity, hypercholesterolemia, a pre-diabetic phenotype and sustained hypertension at 20-weeks. While LVEF was preserved, comorbidities led to LV concentric hypertrophy and diastolic dysfunction as measured by E/e′ and left atrial remodeling, elevated filling pressures, pulmonary hypertension and venous congestion. Endothelial-dependent vascular dysfunction was present in the HFpEF group along with histopathological abnormalities including cardiac fibrosis, adipose infiltrate, pulmonary remodeling, and extensive renal fibrosis.

Conclusions—The combination of WD and DOCA-salt induced hypertension in the Gottingen miniswine led to the development of a new preclinical large animal model of HFpEF exhibiting multi-organ involvement and a full spectrum of comorbidities associated with human HFpEF.

A lack of preclinical large animal models of HFpEF which recapitulate the comorbid-laden syndrome has led to the inability to tease out mechanistic insights and test therapeutic strategies. Herein, we developed a large animal model integrating multiple co-morbid determinants of HFpEF in a miniswine breed that exhibits sensitivity to obesity, metabolic syndrome and vascular disease with overt clinical signs of heart failure. The combination of WD and DOCA-salt induced hypertension in the Gottingen miniswine led to the development of a new large animal model of HFpEF exhibiting multi-organ involvement and a full spectrum of comorbidities associated with human HFpEF.

### ABBREVIATIONS

CVP Central Venous Pressure

DBP Diastolic Blood Pressure

DOCA 11-Deoxycorticosterone Acetate

EF Ejection Fraction

HFpEF Heart Failure with Preserved Ejection Fraction

IVGTT Intravenous Glucose Tolerance Test

LAFAC Left Atrial Fractional Area of Change

LV Left Ventricle

MAP Mean Arterial Pressure

PASP Pulmonary Systolic Pressure

PCWP Pulmonary Capillary Wedge Pressure

PW Pulse Wave

SBP Systolic Blood Pressure

WD Western Diet

### INTRODUCTION

Heart failure with preserved ejection fraction (HFpEF) has emerged as cardiovascular medicine's most complex, comorbidity-laden, heterogenous disease. The increasing prevalence of HFpEF in an obese, diabetic, hypertensive, aging population coupled with lack of effective treatments further complicates this critical public health problem(1). Though complex in its multi-organ pathophysiology, all patients suffering from HFpEF exhibit increased left ventricular filling pressure and reduced exercise tolerance with a preserved left ventricular ejection fraction(2,3). Despite the clear cardiac indices, it is the extra-cardiac comorbidities that are integral in disease progression and actively contribute to the syndrome of HFpEF(4). While there are several shared comorbidities between heart failure with reduced ejection fraction (HFrEF) and HFpEF patients, the higher burden of a subset of comorbidities associated with HFpEF results in a higher risk of mortality(5). Within the growing number of cardiac and noncardiac comorbidities, the most common risk factors/comorbidities associated with HFpEF are age, female gender, hypertensions, renal impairment, diabetes and obesity(5).

The pathophysiological mechanisms involved in disease progression of HFpEF have yet to be clearly defined due to the lack of robust and translationally relevant animal models. The multiple phenotypes in HFpEF has proven difficult to recapitulate in an animal model. Typically, a single organ-single stressor approach is usually favored when developing animal models making the multiple comorbidities associated with HFpEF difficult to mimic in the laboratory(4). One rodent model which has overcome this limitation and has shown to recapitulate several comorbidities through genetic selection is the ZSF-1 obese rat. The ZSF-1 rat model, which crosses the spontaneous hypertensive heart failure rat with a Zucker diabetic fatty rat, provides an overlapping genetic phenotype which predisposes the animals to hypertension and metabolic syndrome, two major prerequisites for the development of HFpEF (6). Several laboratories have demonstrated abnormalities in these animals the parallel human HFpEF (6,7). Various species and models have been used to model concentric LV hypertrophy, a hallmark of HFpEF, though none perfectly resemble the complex myocardial milieu seen with HFpEF. The hypertrophic, fibrotic and impaired relaxation phenotype has been successfully modeled in dogs by repeated coronary microembolization(8) and in aged hypertensive dogs by renal wrapping(9); however, due to societal intolerance for canine experimentation and a growing number of phenotypically-selective breeding and genetically-modified pigs available, swine represent a more suitable large animal for HFpEF model development.

Swine models of HFpEF have been described with varying success using the most prominent amongst the comorbidities: hypertension, obesity and diabetes mellitus generated typically as a single-insult or as a two-hit approach. Models that utilize hypertension as the sole-driver of HFpEF pathophysiology include those that focus the hypertension directly to the myocardium by mechanical manipulations to facilitate pressure overload left ventricular hypertrophy. Devices constricting the aorta with bands(10,11), cuffs(12,13) and stents(14), mitral regurgitation valve chordae rupture(15) and renal artery stenosis combined with ventricular pacing(16) all result in increased myocardial mass, stiffness and fibrosis; however, the absence of multi-organ pathogenic effects significantly limit their clinical applicability. In contrast, pharmacological approaches to induce LV hypertrophy in swine through systemic hypertension by vasopressor(17-19) lack the severity to meaningfully impact cardiac function and fail to demonstrate overt heart failure at rest. Additionally, efforts to incorporate obesity and diabetic comorbidities has been performed through Western diets (WD) high in fat, fructose and salt either alone or in combination with additional stressors in healthy swine(19-22) and strains predisposed to exhibit metabolic syndrome(23,24) and yet these models have failed to faithfully mimic the human HFpEF condition.

Unlike humans, many wild-type animal strains are highly resistant to diabetes, hypertension, atherosclerosis, and fail to exhibit cardiovascular diseases following prolonged exposure to risk factors(25-27). Utilizing animals predisposed to cardiovascular disease provides an optimal background on which exposure to a WD and hypertension will ultimately induce HFpEF. Gottingen miniswine, bred for small size and ease of handling in a laboratory setting, were developed by crossbreeding the Vietnamese, Hormel, and German improved Landrace swine(28). The Gottingen miniswine are described in relation to glucose metabolism and obesity, with propensity to develop various aspects of the metabolic syndrome including obesity, insulin resistance, and glucose intolerance when fed various high fat, WDs(28-30). The Gottingen miniswine also exhibits a propensity for development of dyslipidemia, vascular disease and atherosclerosis(29). Though diabetes and obesity are contributing comorbidities, hypertension is critical in driving the pathophysiological HFpEF phenotype of concentric hypertrophic LV remodeling, myocardial fibrosis and impaired ventricular relaxation.

Using a multi-hit minimally invasive approach, we endeavored to create a clinically relevant miniswine model that combines three common comorbidities (metabolic syndrome, hypertension, female sex) that contribute to the complex pathophysiology of human HFpEF. Superimposing systemic hypertension using deoxycorticosterone acetate (DOCA) onto diet-induced obesity and a pre-diabetic phenotype in female Gottingen miniswine results in a new animal model that exhibits the full spectrum of comorbidities and multi-organ involvement associated with HFpEF disease progression.

Methods

Experimental Design

All animal experiments were performed in accordance with the Guide for the Care and Use of laboratory Animals, the Public Health Service Policy on the Humane Care and Use of Laboratory Animals, and the Animal Welfare Act. Institutional Animal Care and Use Committee (IACUC) approval was obtained from the Louisiana State University Health Sciences Center—New Orleans prior to initiation of these experimental studies.

Fourteen-month old, intact female Gottingen minipigs (17 to 20 kg) were acquired (Marshall BioResources, Rose, N.Y.) and assigned to 2 groups: healthy Control (n=3) and WD+deoxycorticosterone acetate (DOCA)-induced HFpEF (n=8). One HFpEF animal succumbed to sudden death of unknown causes 9 weeks; two HFpEF animals exhibiting severe end-organ damage, lethargy and poor overall health were humanely euthanized at 10 weeks and these animals were not included in the final data analysis. Adjustments to amounts of HFpEF diet and DOCA were implemented with final constituents detailed below and final outcome measures in the HFpEF group assessed in a total of 5 animals.

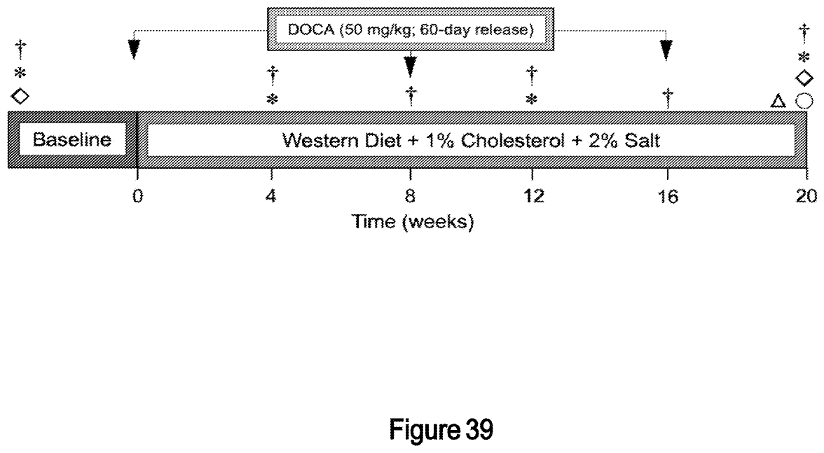

The Control group were fed a standard diet (8753, Teklad Miniswine Diet, Envigo; 2.3 kcal/g-1; carbohydrate: 33%; protein: 16%; fat: 3.9%: cholesterol: 0% wt/wt; salt, sodium chloride: 0.4%; 900 g/day), whereas the HFpEF group was fed 50/50 (wt/wt) mix of standard diet and custom WD containing high levels of fat, fructose, cholesterol and salt (9GZC TestDiet, St. Louis, Mo.: Ossabaw atherosclerotic diet type 5B4L w/ 4% total salt; 4.01 kcal/g-1; carbohydrate: 39.9% [17.8% high-fructose corn syrup]; protein: 16.2%; fat: 40%; cholesterol: 2%; sodium cholate: 0.7%; 900 g/day). Animals were fed once per day and water was provided. Minipigs in the HFpEF group received a subcutaneous DOCA depot (50 mg/kg, 200 mg pellets, 60-day release, Innovative Research of America, Sarasota, Fla.). Altogether, Gottingen miniswine in the Control group (n=3) and in the HFpEF group (n=5) were on-study for a total of 20 weeks (FIG. 39).

Blood Collection

Venous blood samples were obtained from anesthetized animals at baseline and at 4, 8, 12, 16 and 20 weeks, processed for serum, snap frozen and stored at −80° C. Total cholesterol (TC), low density lipoprotein (LDL), and high density lipoprotein (HDL) levels were measured (IDEXX Laboratories, Memphis, Tenn.)

Transthoracic Echocardiography

At baseline, 4, 8, 12, 16 and 20 weeks, transthoracic echocardiography (Vivid E9, M5S transducer, GE Healthcare, Wauwatosa, Wis.) was performed in miniswine under ketamine/xylazine induction anesthesia (15 mg/kg/1.5 mg/kg, IM) and then isoflurane anesthesia (1.5% in 100% oxygen) as maintenance. To ensure accurate and consistent image acquisition, diltiazem (0.25 mg/kg) was administered to reach a target heart rate <90 bpm(31). Left ventricular (LV) volumes and ejection fraction was measured using 2D auto EF. Auto EF utilizes speckle tracing technology and Simpson's method to track the endocardium through systole and diastole(32). LV wall thickness at end-systole and end-diastole were measured from subcostal 2D B-mode images acquired at the level of the mitral valve leaflets as previously described (32). Left atrial (LA) areas and fractional area of change were measured from subcostal 2D B-mode views (33).

Pulse wave (PW) doppler echocardiography, with a maximal corrected angle of 40°, was used to assess transmitral inflow velocities during diastole at the level of the mitral valve orifice. The ratio of peak early (E) and late (A) transmitral velocities was calculated. PW tissue doppler imaging, with a maximal corrected angle of 40°, was used to calculate tissue velocities during early ventricular filling (e′) at the mitral valve medial and lateral annulus. Medial and lateral ratios of early transmitral inflow and tissue velocity (E/e′) were calculated(33). All images were acquired by a single cardiovascular researcher (TES) experienced in miniswine echocardiography. Analysis were performed offline in a blinded fashion (GE EchoPAC Software, Version 202, GE Healthcare, Wauwatosa, Wis.).

Invasive Hemodynamics

Miniswine underwent invasive systemic, left ventricular, and pulmonary hemodynamic measurements at baseline and at 20 weeks. Animals were sedated as above, intubated, ventilated and maintained under methohexital (Brevital, 7.0-8.0 mg/kg/hr, IV) as previously described(32,34,35). Electrocardiogram, heart rate, respiration rate, 02 saturation, arterial blood pressure and body temperature were continuously monitored. Using standard sterile technique, percutaneous femoral artery and vein sheath introducers were placed under ultrasound guidance (Vivid E9, ML6-15 transducer, GE Healthcare, Wauwatosa, Wis.). Systemic arterial blood pressures (SBP, DBP, MAP) were obtained using from femoral artery access and recorded on the TruWave Pressure Transducer® (Edwards Lifescience, Irvine, Calif.). For left ventricular hemodynamics, a solid state single-pressure catheter (Millar Instruments, TX) was advanced through the ascending thoracic aorta via femoral artery access under fluoroscopic guidance (Optima CL232i; GE Healthcare Wauwatosa, Wis.) and subsequently placed in the LV chamber for recording of LV pressures. Steady-state data was recorded (PowerLab 8/35, ADInstruments, Colorado Springs, Colo.) under spontaneous heart rate for a minimum of 10 consecutive beats with the ventilator tidal volume set to zero to eliminate respiratory artifact. Right heart catheterization was performed with a Swan-Ganz catheter (Edwards Lifesciences, Irvine, Calif.) placed in the left branch of the pulmonary artery via femoral vein access. Central venous pressure (CVP), pulmonary artery systolic, diastolic, mean and wedge pressure (PASP, and PCWP, respectively) were recorded (TruWave Pressure Transducer, Edwards Lifescience, Irvine, Calif.). Pressure measurements were performed offline in a blinded fashion (LabChart 8 Software, ADInstruments, Colorado Springs, Colo.).

Central Venous Line Placement and Intravenous Glucose Tolerance Testing

For intravenous glucose tolerance test (IVGTT) serial blood sampling, minipigs were sedated, anesthetized using isoflurane as above and an indwelling catheter (Hickman, C. R. Bard, Inc, Salt Lake City, Utah) surgically placed in the right jugular vein as previously described(34,35). Surgical complications with implantation of one indwelling catheter led to only four HFpEF animals undergoing the IVGTT. Animals were recovered for 3 days prior to IVGTT. At 20 weeks, conscious, overnight fasted miniswine received an intravenous bolus of 50% glucose solution (0.5 g/kg body weight, Animal Health International, Patterson, Colo.) and blood samples (3 ml) collected at −5, 0, 2.5, 5, 10, 20, 30, 40, 50, 60 min. Blood glucose levels were monitored (Contour Blood Glucose Monitoring System, Bayer Healthcare, Mishawaka, Ind.) and plasma insulin values were measured (Insulin ELISA, Mercodia, Winston-Salem, N.C.).

Euthanasia

At 20 weeks, minipigs were sedated, intubated, and anesthetized as above. Heparin (300 U/kg IV) administered and under deep anesthesia, minipigs were euthanized (potassium chloride, KCl, 40 mEq/kg IV, Hospira, Inc, Lake Forest, Ill.) in accordance with the 2013 Edition of the AVMA Guidelines for the Euthanasia of Animals.

Ex Vivo Coronary Vascular Reactivity

Hearts were excised, and the left anterior descending (LAD) and circumflex (LCX) coronary arteries carefully dissected, cut into 3-5 mm rings and mounted onto the tension apparatus within organ bath chambers (Radnoti, Glass Technology, Monrovia, Calif.) with oxygenated Krebs buffer for isometric tension experiments as previously described(32,36). Briefly, LAD and LCX coronary arterial rings were placed under 2 grams of preload tension and allowed to stabilize for 60-90 min. Coronary arterial ring viability was assessed using 50 and 100 mM KCl consecutively. Rings were washed, precontracted with prostaglandin (PGF2a, 30 μM) and endothelial-dependent relaxation concentration curves were generated using bradykinin (10¹¹ to 10⁻⁶ M) and substance P (10⁻¹² to 10⁻⁸ M). Endothelial-independent relaxation concentration curves were generated using sodium nitroprusside (SNP, 10⁻⁹ to 10⁻⁵M). Maximal relaxation and half maximal effective concentration (EC50) were calculated.

Circulating Biomarkers of Cardiovascular Disease Risk

To assess oxidative stress in our model we measure 8-isoprostane levels in plasma from baseline, 4, 12 and 20 weeks in all animals using an enzyme-linked immune-assay kit (Cayman Chemical Company, Ann Arbor, Mich.). Endothelin-1 has been well established as a marker of endothelial dysfunction (37). Circulating serum levels of endothelin-1 were measured at baseline, 4, 12 and 20 weeks in all animals using an enzyme-linked immune-assay kit (Enzo Lifescience, Farmingdale, N.Y.).

RNA Isolation and PCR Analysis of Natriuretic Peptides

Real-time PCR (qPCR) mRNA levels of left ventricular expression of natriuretic peptides were assessed as previously describe (32). Briefly, mRNA was isolated from left ventricular tissue and gene expression of atrial natriuretic peptide (ANP) and B-type natriuretic peptide (BNP).

Fibrosis Staining and Quantification

Heart, lung, liver and kidney specimens were harvested, fixed in 10% neutral buffered formalin, processed for paraffin embedding, sections cut (5 μm), stained with Masson's trichrome and photomicrographs acquired. Left ventricular, pulmonary, hepatic and renal cortical fibrosis was quantified by calculating the percentage of total Masson's trichrome positive tissue (blue) over the total tissue area using image J software. Hepatic lobule area was quantified by identification of the central vein and portal triads along the perimeter with an outline drawn around the perimeter and through the portal triads to acquire hepatic lobule area.

Statistical Analysis

All data are expressed as the mean±standard error of the mean (SEM). Statistical analyses were performed using Prism 6 (GraphPad Software, San Diego, Calif.). Repeated 2-way analysis of variance (ANOVA) was performed for multiple comparisons between two groups over time with a Sidak post-hoc test correction for multiple comparisons. A Student unpaired, 2-tailed, τ test was performed when comparing data from two groups at a single time point. A p-value of <0.05 was considered statistically significant.

Results

Gottingen Minipigs' Enhanced Sensitivity to Multiple Cardiovascular Disease Insults

Based on previous studies using WD-induced obesity in Gottingen minipigs(28,29), Gottingen miniswine were fed full rations of the custom WD containing 2% cholesterol and 4% sodium chloride (9GZC TestDiet, St. Louis, Mo.). Since DOCA-induced hypertension had not been previously performed in Gottingen miniswine, we based our initial DOCA implantation regimen of 100 mg/kg, 90-day release pellets on reported literature using Landrace pigs(20). Following 4 weeks of WD diet and DOCA (100 mg/kg, 90-day), serum cholesterol levels increased 20-fold (data not shown) and Gottingen miniswine (n=3) were returned to standard control diet. Though cholesterol levels were improved when placed on standard diet, it appeared that the high levels of DOCA contributed to the moribund condition of the pigs and consequently, one Gottingen minipig was lost due to sudden death and the other two minipigs were humanely euthanized due to severe end-organ damage. As a result of the early mortalities, animals in the HFpEF group were subsequently fed a modified custom WD (50/50 wt mix with standard control diet [HFpEF diet]) and implanted with DOCA at 50 mg/kg, 200 mg pellets, 60-day release.

Gottingen Minipigs Exhibit Obesity and Hypercholesterolemia

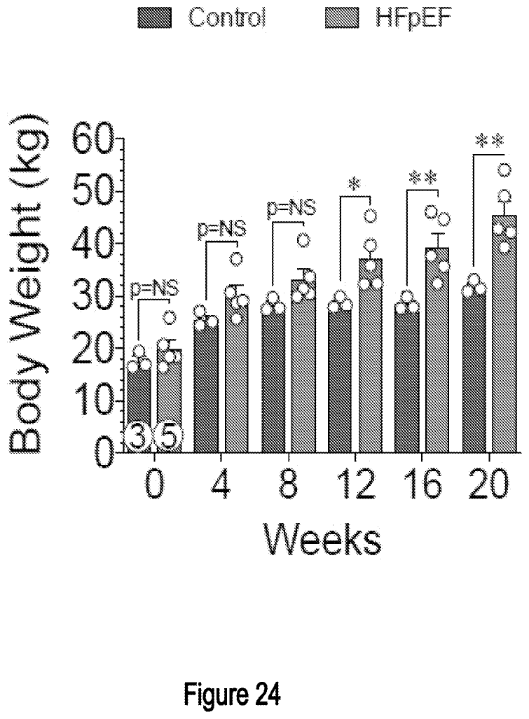

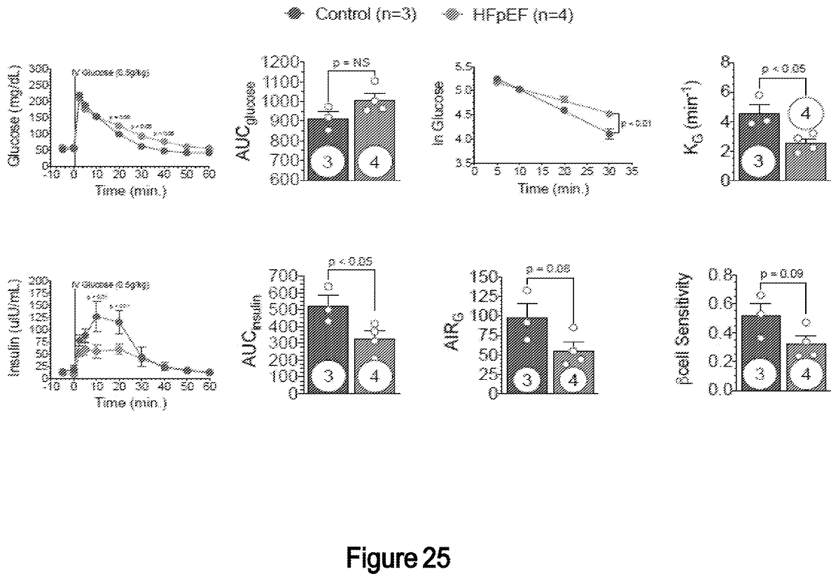

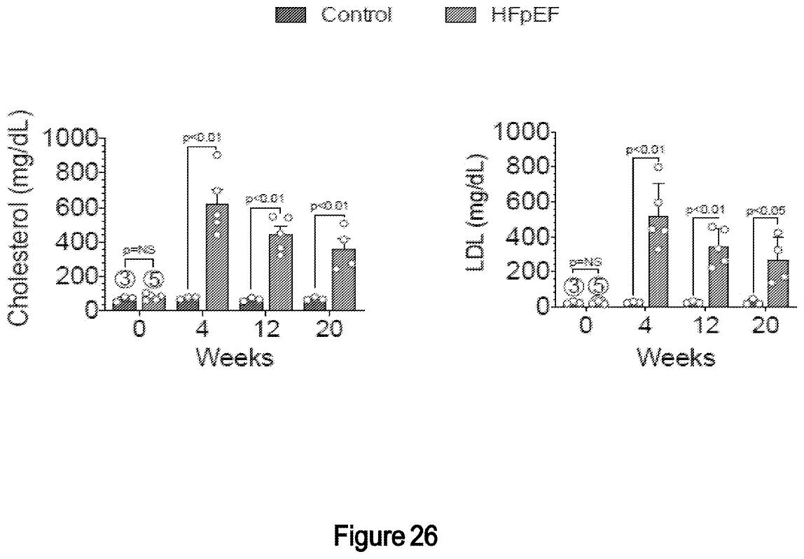

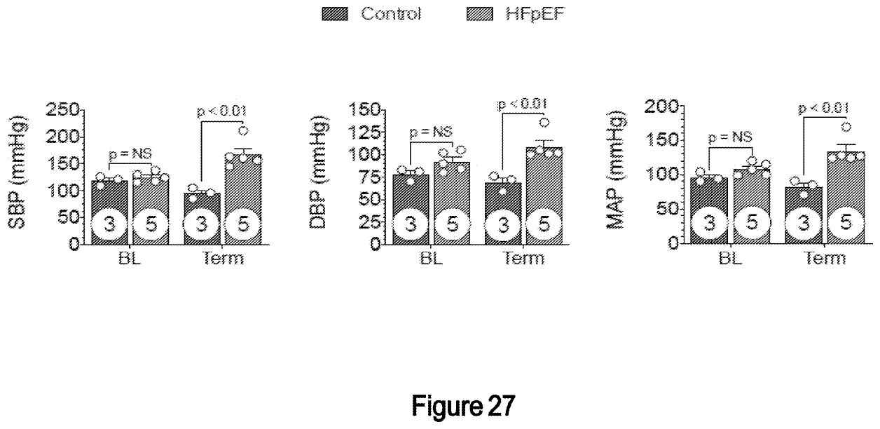

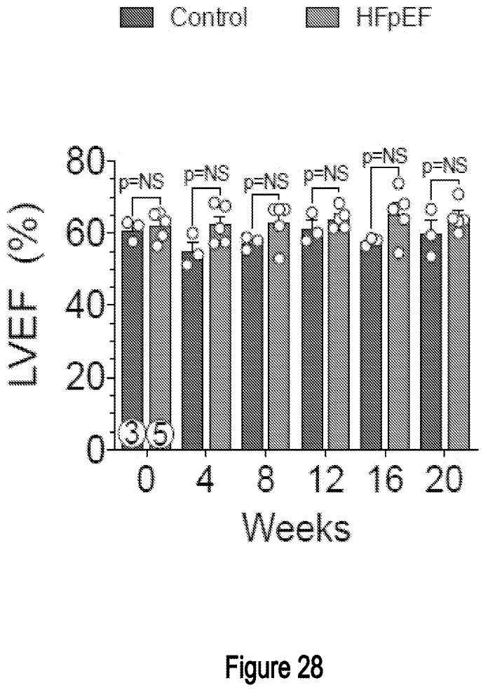

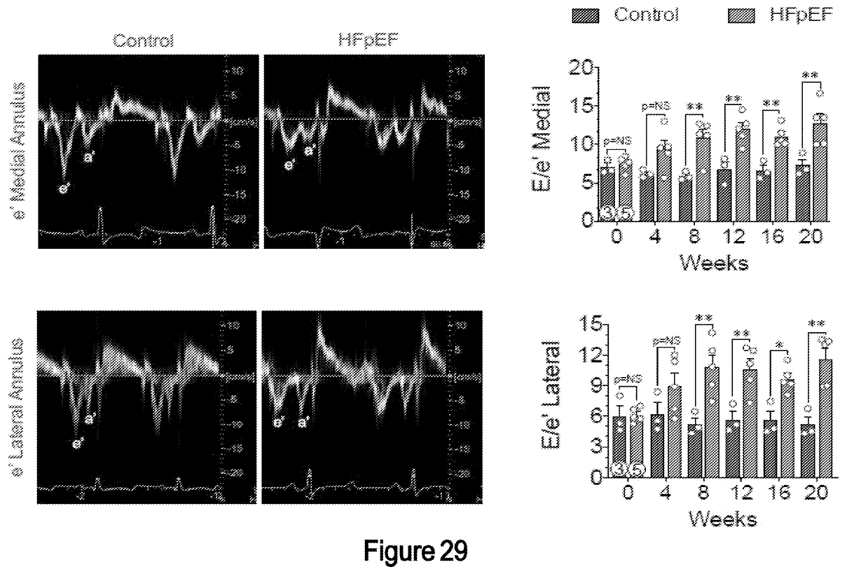

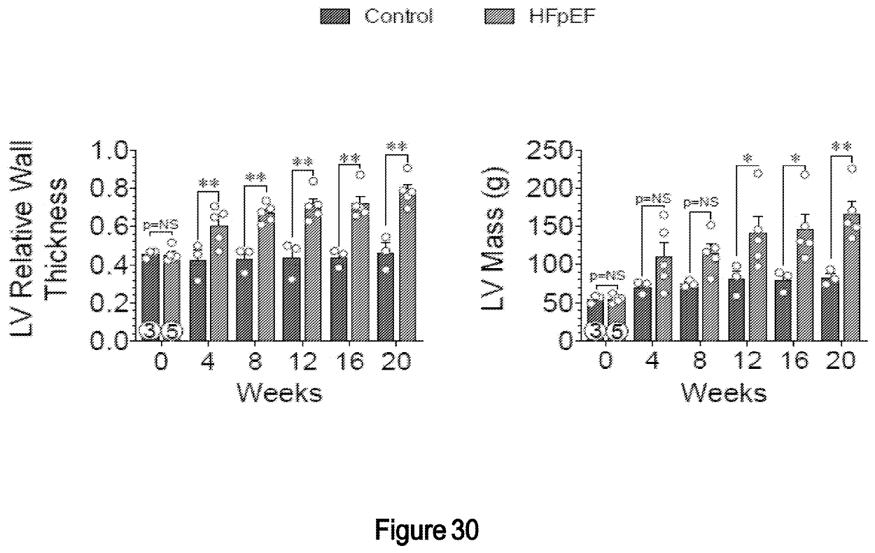

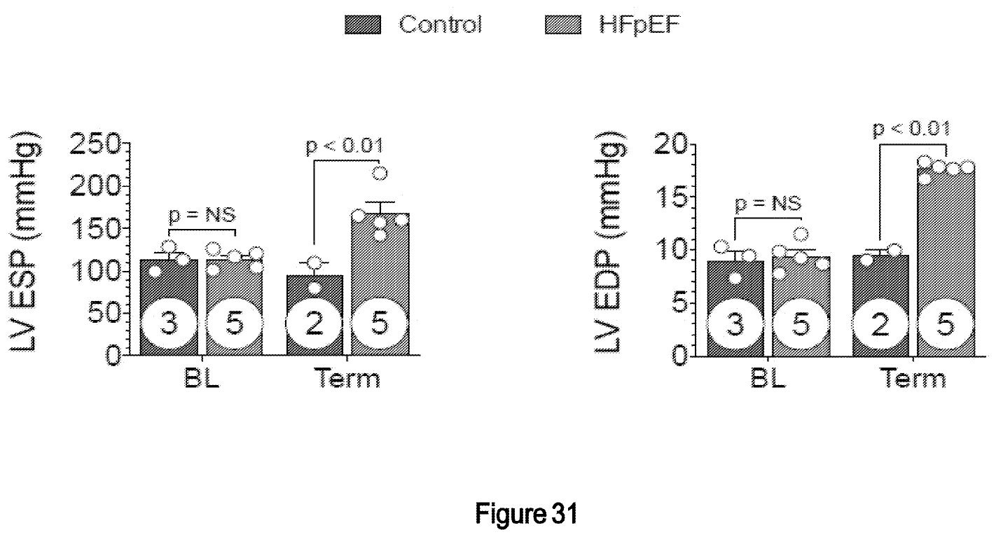

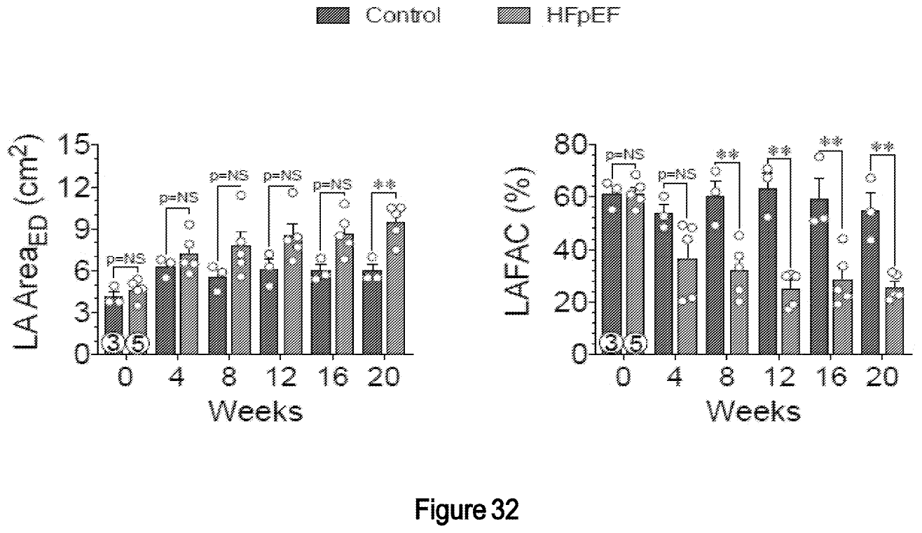

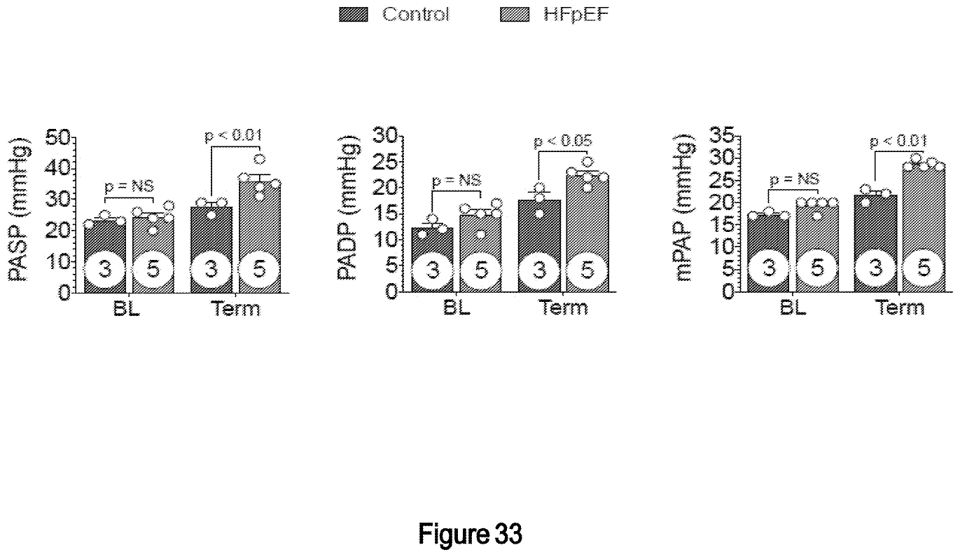

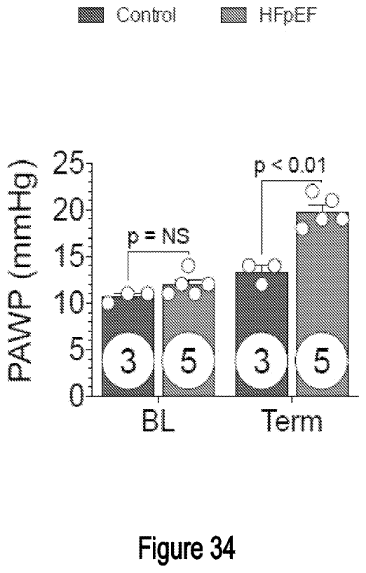

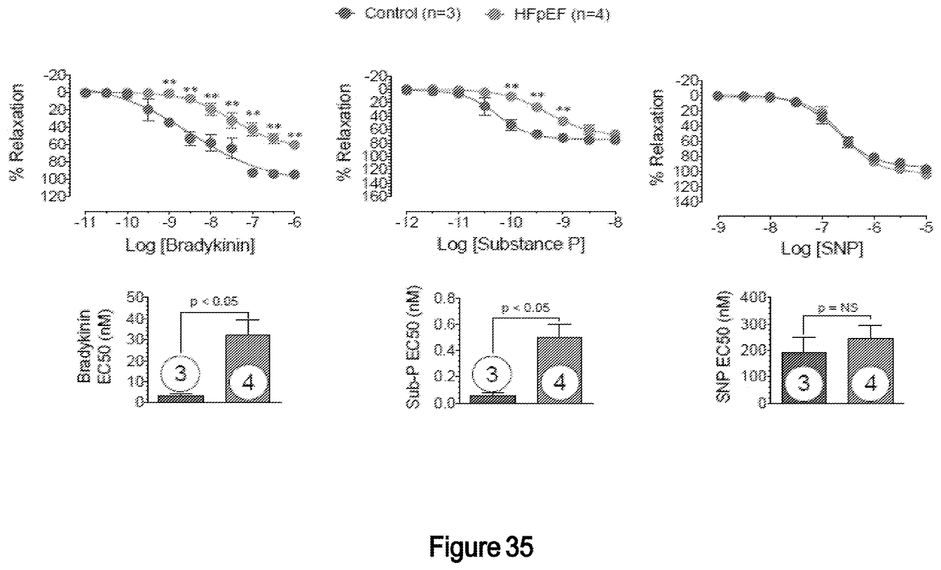

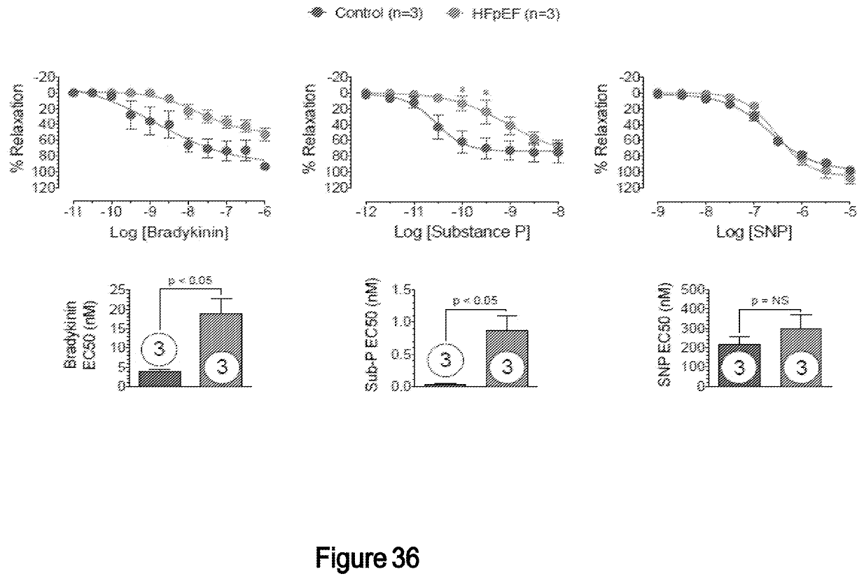

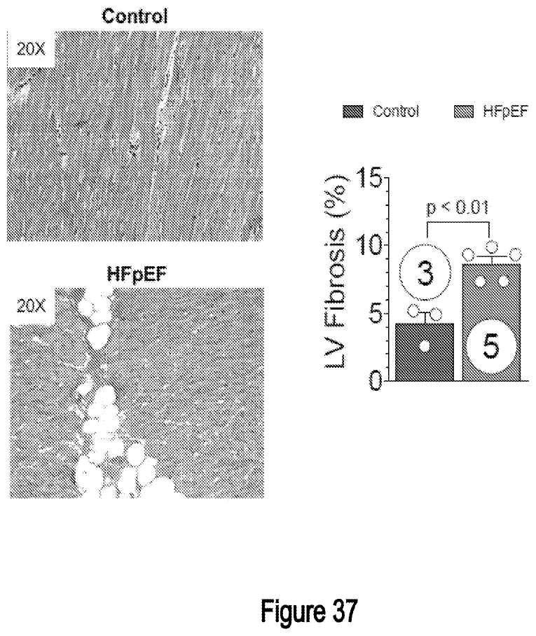

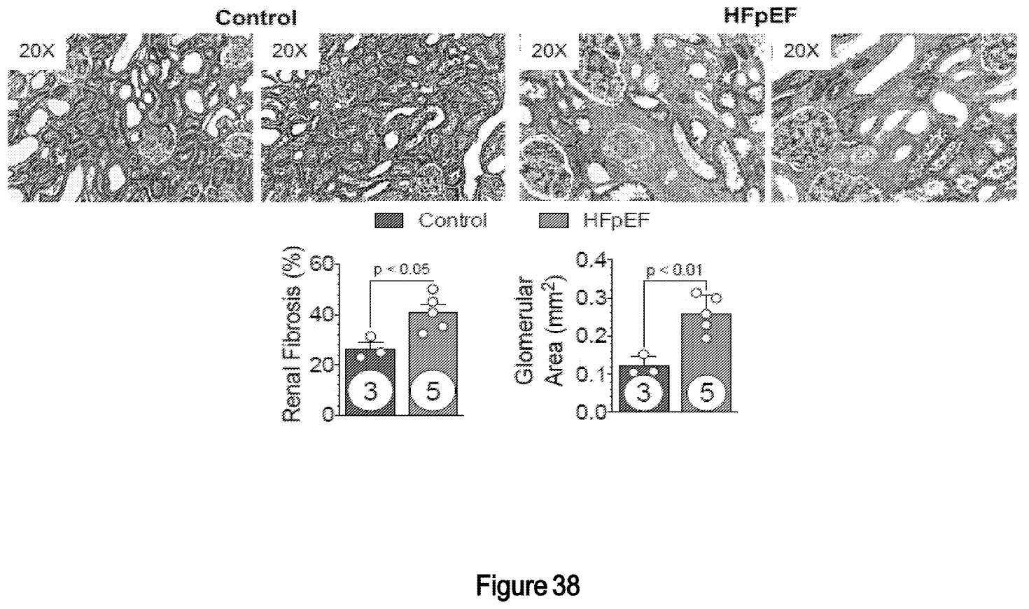

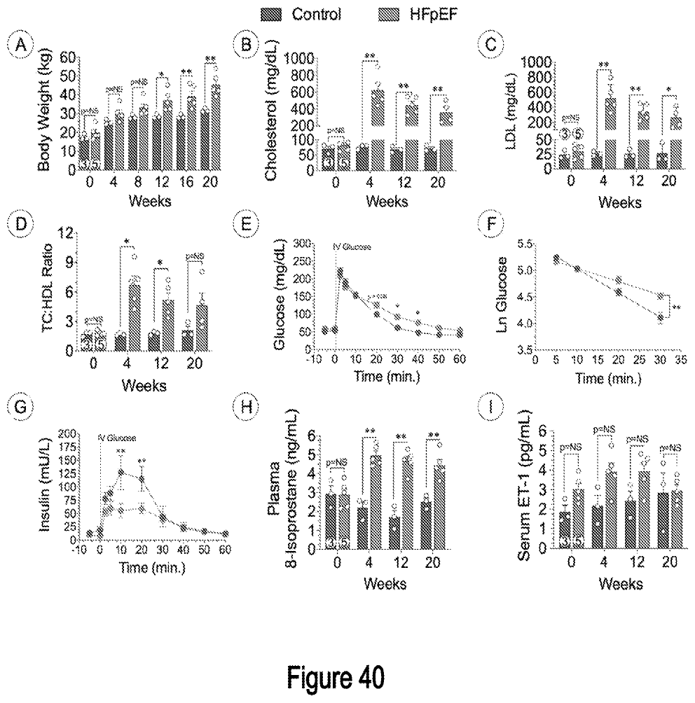

Minipigs fed the HFpEF diet for 20 weeks gained significant (p<0.05) weight starting at 12 weeks compared to control diet fed animals (FIG. 24). At 20 weeks, HFpEF animals weighed 45.4±2.7 kg compared to control 31.7±0.7 kg (p<0.01) (FIG. 40, Panel A). To confirm hypercholesterolemia, we performed serial blood draws and measured TC, LDL and HDL. At 4 weeks, TC levels in the HFpEF animals was significantly (p<0.01) increased compared to control (623±81.6 mg/dL vs. 77±4.0 mg/dL, respectively) (FIG. 40 Panel B). TC was significantly elevated throughout the 20-week study in HFpEF animals compared to control (p<0.01) (FIG. 40 Panel B). As shown in FIG. 40 Panel C, circulating LDL cholesterol levels exceeded 200 mg/dL during the study and were significantly elevated (p<0.01, 4, 8 weeks; p<0.05, 20 weeks) compared to controls. The TC:HDL ratio, a clinical index and independent risk predictor of heart disease was calculated (FIG. 40 Panel D). At 4 and 12 weeks, the TC:HDL ratio was significantly (p<0.05) elevated in HFpEF animals compared to control; though was not significant at the 20 week timepoint.

Altered Glucose Metabolism and Circulating Insulin Levels in Response to IVGTT

Minipigs underwent intravenous glucose tolerance testing at 20 weeks to determine systemic responsiveness towards a glucose metabolism (FIG. 40 Panels E-G). Baseline fasting blood glucose and insulin levels were similar between control and HFpEF animals at 20 weeks. Following intravenous administration of 0.5 g/kg of glucose, circulating levels increased to >200 mg/dL in all animals. At 30 and 40 minutes, post-IV glucose administration, circulating levels were significantly higher in HFpEF animals compared to controls (p<0.05) (FIG. 40 Panel E). When calculating the natural log of glucose over 5-30 min, there was a significant (p <0.01) reduction in the clearance of glucose over time (FIG. 40 Panel F). Basal insulin levels were not different between groups and following IV glucose administration, circulating insulin was significantly blunted in the HFpEF group between 10 and 30 min (p<0.01) (FIG. 40 Panel G).

Biomarkers of Cardiovascular Disease Risk

Oxidative stress is a driving force of long-term abnormalities in cardiovascular disease. 8-isoprostane was measure in the plasma in all animals at baseline, 4, 12, and 20 weeks in the study (FIG. 40 Panel H). At baseline there was no significant difference between groups, as early as 4 weeks and throughout the entirety of the study 8-isoprostane levels were significantly (p<0.01) elevated in the HFpEF group compared to the control group (FIG. 40 Panel H). Endothelin-1, a marker of endothelial dysfunction in cardiovascular disease including coronary artery disease and peripheral artery disease, was measured at baseline, 4, 12 and 20 weeks. While elevated throughout the study at 4, 12, and 20 weeks in the HFpEF group compared to control, there was no statistical difference between the groups (p=NS) (FIG. 40 Panel I).

DOCA- and Salt-Induced Hypertension

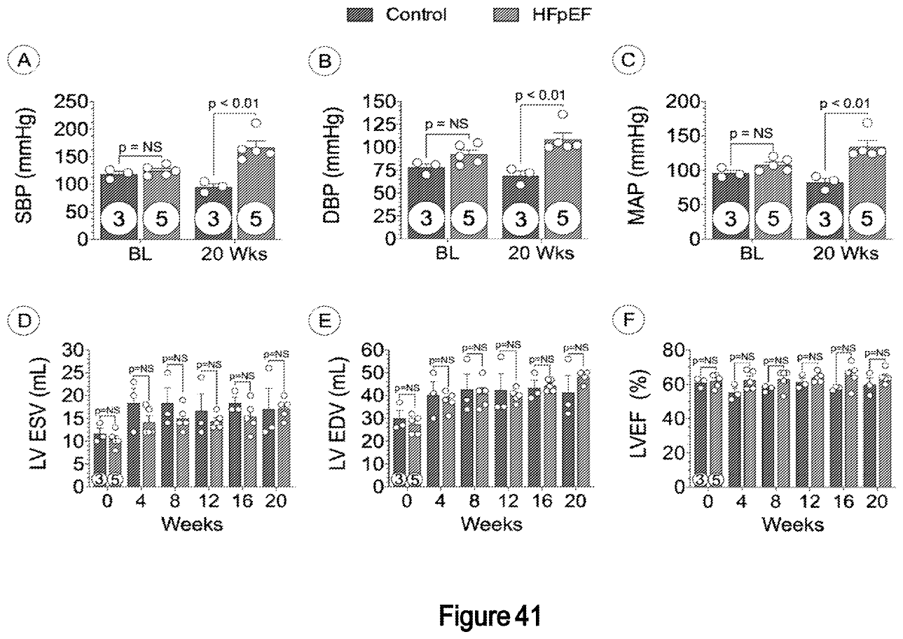

DOCA pellet implantation (50 mg/kg, 60-day release) and increased dietary salt intake in HFpEF miniswine resulted in significant elevations in systemic blood pressures at 20 weeks (FIG. 41). Arterial invasive hemodynamic measurements were obtained at baseline and at 20 weeks. Systolic blood pressures were similar at baseline but were significantly higher at 20 weeks in the HFpEF group compared to controls (167±11.4 vs. 95±5.8 mmHg, respectively; p<0.01) (FIG. 41 Panel A). Diastolic blood pressures were not different at baseline but were significantly higher at 20 weeks in the HFpEF group compared to controls (109±6.9 vs. 69±5.1 mmHg, respectively; p<0.01) (FIG. 41 Panel B). Mean arterial pressure at 20 weeks was significantly higher in HFpEF animals (134±8.7 vs. 82±6.0 mmHg [control]) (FIG. 41 Panel C).

Preserved LV Systolic Function with Progressive Diastolic Dysfunction

Echocardiograms acquired every 4 weeks revealed that systolic LV function was maintained over the 20-week study (FIG. 41 Panels D-F). There were no significant differences in end-systolic or end-diastolic volumes between HFpEF and control minipigs (FIG. 41 Panels D & E). Importantly, LVEF was unchanged throughout the 20-week study in HFpEF and control minipigs (FIG. 41 Panel F).

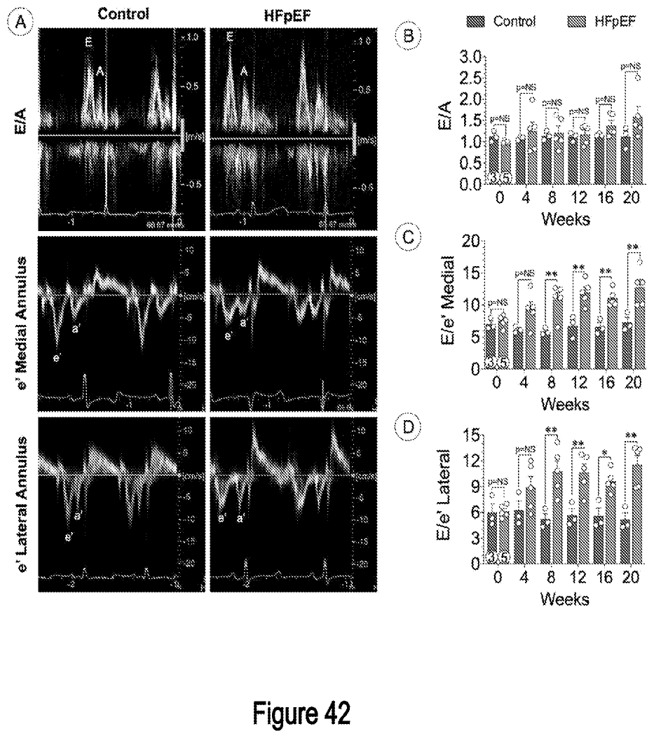

Diastolic function was assessed by measuring mitral valve inflow velocity using pulse wave doppler and tissue doppler echocardiography of the medial and lateral mitral valve annulus (FIG. 42). Representative images of mitral inflow velocities (top row) and tissue doppler (bottom rows) from control and HFpEF minipigs at 20 weeks are shown (FIG. 42). During the 20-week study, mitral valve inflow velocity (E/A ratio) was not significantly different between control and HFpEF minipigs (FIG. 42 Panel B); however, the ratio of early inflow velocity (E) over tissue velocity (e′) was significantly elevated in the HFpEF minipigs starting at 8 weeks and remained elevated at all subsequent timepoints (FIG. 42 Panels C and D). By 8 weeks, the medial E/e′ ratio (FIG. 42 Panel C) was significantly elevated in HFpEF minipigs compared to controls (p<0.01) and was sustained over the course of the study (20 week: HFpEF, 12.8±1.3; Control, 7.3±0.8; p<0.01). By 8 weeks, the lateral E/e′ ratio (FIG. 42 Panel D) was significantly elevated in HFpEF minipigs compared to controls (p<0.01) and was sustained over the course of the study (20 week: HFpEF, 11.5±1.1; Control 5.2±0.8; p<0.01).

Increased LV Wall Thickness and Concentric Hypertrophy

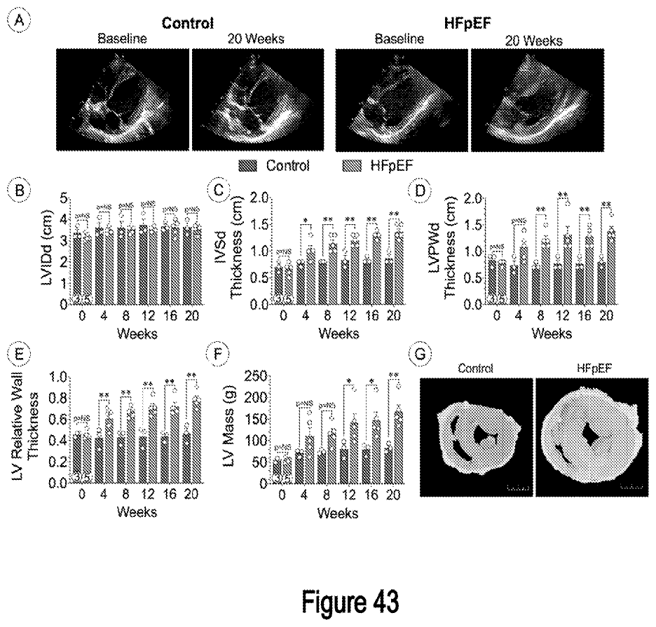

Serial echocardiographic assessment of left ventricular structure showed progressive and significant thickening of the LV walls in HFpEF miniswine (FIG. 43). In FIG. 43 Panel A, representative subcostal 2D B-mode images of control (left panels) and HFpEF (right panels) hearts at baseline and 20 weeks are shown. LV chamber dimensions did not change between groups throughout the study (FIG. 43 Panel B). As early as 4 weeks, there was a significant and sustained (p<0.05) increase in end-diastolic intraventricular septal wall thickness (FIG. 42 Panel C) in HFpEF minipigs compared to controls (20 week: 1.36±0.06 vs. 0.87±0.07 cm, respectively; p<0.01). As shown in FIG. 43 Panel D, there was a significant and sustained increase in LV end-diastolic posterior wall thickness starting at 8 weeks (p<0.01). Baseline relative wall thickness (FIG. 43 Panel E) were similar between groups; however, beginning at 4 weeks, there was a significant increase in relative wall thickness in the HFpEF minipigs compared to controls (0.61±0.04 vs. 0.42±0.06 cm, respectively; p<0.01) and continued to increase over the course of the study (20 week: HFpEF, 0.79±0.03 cm; Control, 0.46±0.05 cm; p<0.01). Calculated echocardiographic left ventricular mass (FIG. 43 Panel F) was similar between groups at baseline, 4 and 8 weeks. At 12 weeks, there was a significant increase in LV mass in HFpEF minipigs compared to controls (141±21 vs. 81±12 g, respectively; p<0.05) and at the 20-week end-point, estimation of LV mass in the HFpEF group was 167±16 g (p<0.01 vs control). In FIG. 43 Panel G, representative photomicrographs of mid-papillary cross-sections of the myocardium from a control and HFpEF heart demonstrate a clear difference in morphometry of the heart.

Elevated LV Filling Pressures with Left Atrial Remodeling and Dysfunction

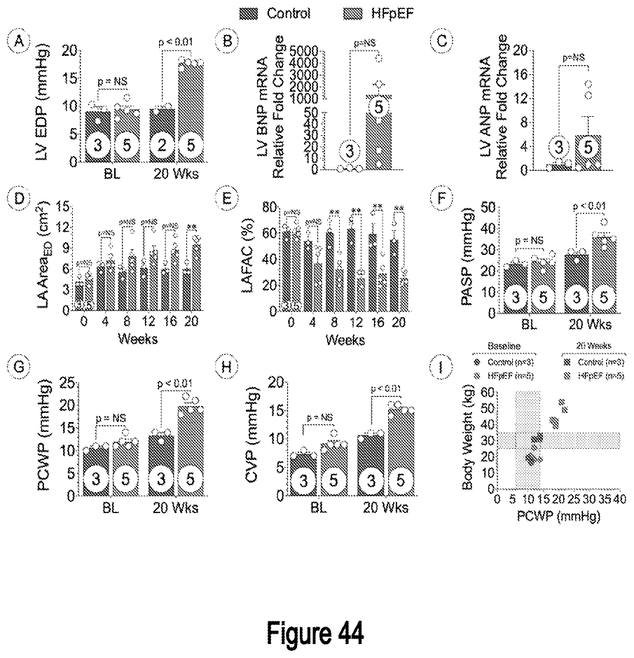

In the HFpEF minipigs, we observed a significant elevation in left ventricular pressures coupled with adverse remodeling and function of the left atria (FIG. 44). Left ventricular pressures were acquired in all animals at baseline and all HFpEF and 2 control minipigs at 20 weeks. In FIG. 44 Panel A, left ventricular end-diastolic pressure (LVEDP) was significantly (p<0.01) increased in HFpEF animals (17.7±0.3 mmHg) at the 20 week timepoint and unchanged in controls (9.0±0.9 [baseline]; 9.5±0.5 [20 weeks], mmHg). In FIG. 44 Panel B, left ventricular mRNA expression of B-type natriuretic peptide was elevated by greater than 4.5-fold in every animal in the HFpEF group compared to control. However, due to variability in the HFpEF group and small sample size, B-type natriuretic peptide expression levels did not demonstrate statistical significance. Left ventricular atrial natriuretic peptide expression was not statistically different between groups (FIG. 44 Panel C). FIG. 44 Panel D demonstrates progressive remodeling of the left atria (LA) as measured using echocardiography of left atrial area at end-diastole (ED). The mean LA area (FIG. 44 Panel D) at 20 weeks was 9.5±0.6 cm² in HFpEF minipigs compared to 6.0±0.5 cm² in control animals (p<0.01). The LA fractional area of change (LAFAC) was significantly reduced in HFpEF minipigs as early as 8 weeks (p<0.01), and continued to deteriorate over time compared to controls (FIG. 44 Panel E).

Pre- and Post-Capillary Pulmonary Hypertension

Right heart catheterization was performed at baseline and 20 weeks to assess pulmonary pressures (FIG. 44). At baseline, there were no significant differences (p=NS) in pulmonary systolic (FIG. 44 Panel F) and wedge pressures (FIG. 44 Panel G) between control and HFpEF groups. At 20 weeks, pre-capillary pulmonary arterial hypertension (PAH) was observed in which there was a significant increase in PASP (FIG. 44 Panel F) in HFpEF minipigs compared to controls (36.0±2.0 vs. 24.4±1.3 mmHg, respectively; p<0.01). As shown in FIG. 44 Panel G, PCWP was significantly elevated (p<0.01) at 20 weeks in HFpEF minipigs compared to controls (19.8±0.7 vs. 12.0±0.6 mmHg, respectively). There was a significant (p<0.01) increase in CVP (FIG. 44 Panel H) at 20 weeks in HFpEF minipigs (15.2±0.4 mmHg). In FIG. 44 Panel I, we observe a positive correlation in body weight and PCWP in HFpEF minipigs.

Impaired Coronary Artery Endothelial Function

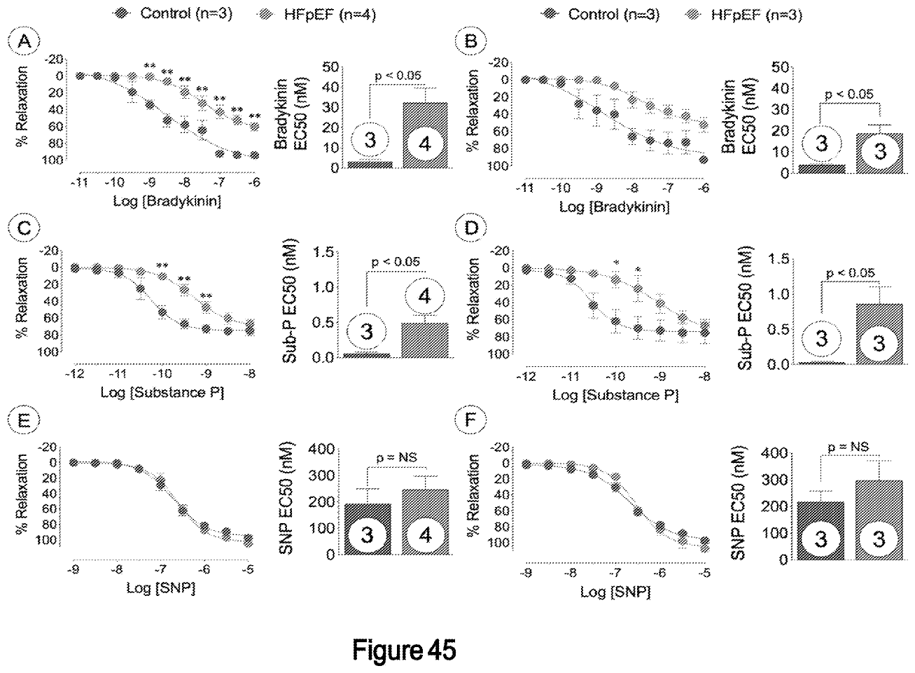

In the setting HFpEF, there is profound coronary artery dysfunction manifested by impaired relaxation to endothelial-dependent agonists(1). At 20 weeks, left anterior descending (LAD) and left circumflex (LCX) coronary arteries were isolated from control and HFpEF Gottingen minipig hearts. Ex vivo vasodilatory responses to endothelial-dependent (bradykinin and substance P) and -independent (sodium nitroprusside) agonists are shown (FIG. 45). LAD coronary arteries from HFpEF minipigs demonstrated significant (p<0.01) attenuation of relaxation to bradykinin (10⁻⁹ to 10⁻⁶M) and significantly greater EC50 compared to controls (FIG. 45 Panel A). Though relaxation response curves to bradykinin were similar, the EC50 was significantly (p<0.05) greater in LCX coronary arteries isolated from HFpEF minipigs compared to controls (FIG. 45 Panel B). Coronary vasodilatory responses to substance P (FIG. 45 Panels C and D) were significantly attenuated at 10⁻¹⁰ to 10⁻⁹M concentrations in LAD (p<0.01) and LCX (p<0.05) and significantly greater EC50 (p<0.05) from HFpEF minipigs compared to controls. Interestingly, endothelial-independent relaxation responses to SNP were similar in LAD and LCX coronary arteries isolated from control and HFpEF minipigs (FIG. 45 Panels E and F).

Multi-Organ Histopathology with Increased LV Myocardial and Renal Fibrosis

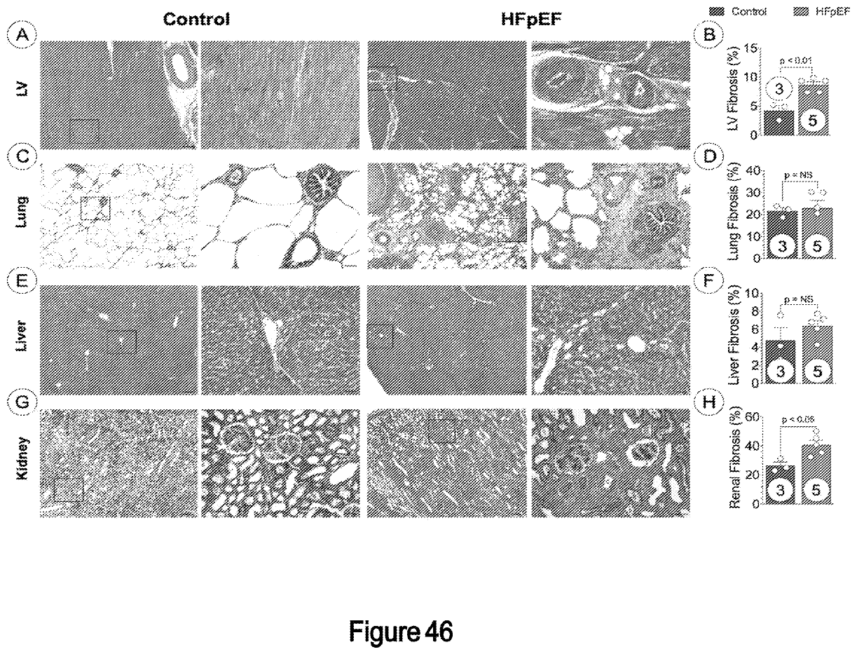

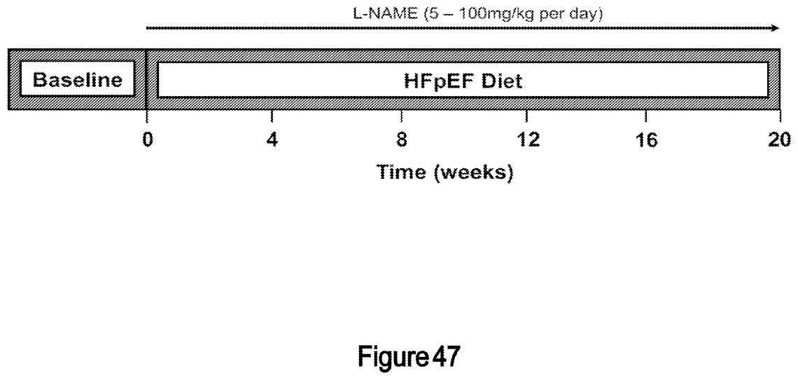

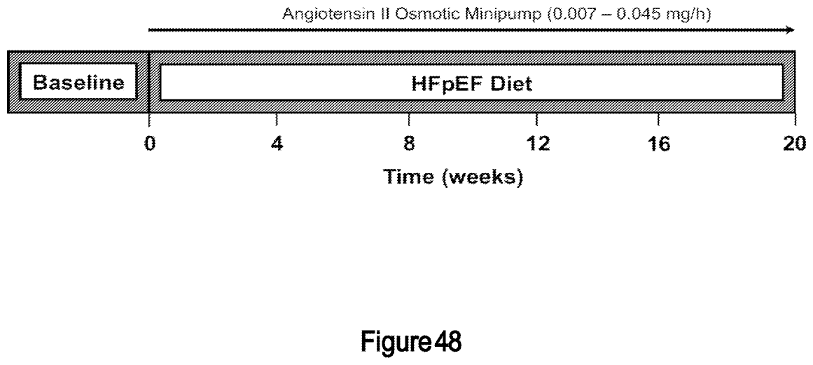

In HFpEF minipigs, representative photomicrographs of left ventricular Masson's trichrome stain demonstrate increased myocardial fibrosis associated with interstitial lipid deposition within the mid-myocardium (FIG. 46 Panel A). When fibrosis was quantified as a percentage of total tissue area, there was a significant (p<0.01) increase in myocardial fibrosis in HFpEF minipig hearts compared to controls (FIG. 46 Panel B). Representative photomicrographs of Masson's trichrome stained lung tissue from control and HFpEF minipigs show a difference in aveolar structure, accumulation of cell infiltrate around pulmonary vasculature and airways (FIG. 46 Panel C). When total pulmonary fibrosis was quantified as a percentage of total tissue area, there was no significant difference (FIG. 46 Panel D). Representative photomicrographs of liver Masson's trichrome stain demonstrate interlobular fibrosis (FIG. 46 Panel E); however, when quantified there was no significant difference between groups (FIG. 46 Panel F). Representative kidney cortical tissue samples from control and HFpEF minipigs show an increase in both glomerular and tubular fibrosis compared to controls (FIG. 46 Panel G). Total renal fibrosis, quantified as percentage of Masson's trichrome stained tissue to total tissue area (FIG. 46 Panel H) was significantly (p<0.05) increased in HFpEF minipigs compared to controls (42±3% vs. 27±3%, respectively).

### DISCUSSION

In this study, we report a large animal model of HFpEF induced through dietary and chronic mineralocorticoid administration using a minipig breed with known susceptibilities toward obesity and metabolic syndrome, and atherosclerosis. Severe LV diastolic dysfunction was evidenced by significant elevations in end-diastolic pressure, diastolic early filling velocities (E/e′), coupled with profound myocardial hypertrophic and fibrosis during which EF was preserved. We observed significant vascular injury and dysfunction evidenced by pulmonary and systemic hypertension as well as impaired coronary artery endothelial-dependent vasorelaxation responses in vitro. In addition to the cardiac pathology, this HFpEF model encompasses impairments across multiple organ systems including pancreas, liver and kidney as exhibited by significant blunting of glucose and insulin handling, elevated circulating cholesterol levels and increased renal fibrosis. The combination of established methods of DOCA-salt induced hypertension and WD metabolic syndrome have not been previously performed in Gottingen miniswine. Our results describe a unique miniswine translational animal model that exhibits the spectrum of multi-organ pathophysiology characteristic of human HFpEF.

There is universal agreement that the lack of suitable preclinical animals models of HFpEF is among the largest roadblocks to advancing our understanding of HFpEF and developing new therapies to treat HFpEF patients. Most of the previous described HFpEF models consist of cardiac pressure-overload to induce LV concentric hypertrophy and diastolic dysfunction, but these models fail to fully capture the characteristics of human HFpEF and have not proven to be reliable for preclinical evaluation of potentially new therapeutic targets(38). Purported animal models of HFpEF exhibit select features of human condition and do not truly reflect the full spectrum of pathological phenotypes observed in HFpEF patients(39). The Gottingen minipig DOCA-salt and WD model described here is to our knowledge the only large animal model to date that exhibits definite evidence of advanced, severe HFpEF. Clinically, diagnostic confirmation of HFpEF requires either elevated LV filling pressures at rest (LVEDP >16 mmHg) or an elevated mean capillary wedge pressure at rest (PCWP >15 mmHg) in the presence of normal systolic LV function (LVEF >50%)(40-43). As we've demonstrated, the Gottingen DOCA-salt and WD model exhibits elevated LV filling pressures at rest (LVEDP, 17.7±0.3 mmHg) and elevated mean capillary wedge pressure at rest (PCWP, 19.8±0.7 mmHg) in the presence of normal systolic LV function (LVEF, 64.3±1.8%). Furthermore, this model is representative of advanced HFpEF as LV filling pressures were elevated at rest, whereas earlier stage HFpEF, is characterized by LV pressure increases observed only during exercise(22). Several other swine models have established comorbid-laden models attempting to mimic HFpEF (24,44); however, they do not demonstrate the clinical endpoints necessary for classification of HFpEF, specifically the non-invasive diastolic echocardiographic measurement of E/e′ and the invasive hemodynamic measurements of elevated LV end-diastolic pressure or elevated PCWP (42,45).

It is well recognized that diastolic dysfunction by itself is not enough to produce HFpEF and additional cardiac and extracardiac abnormalities involving multiple organs are critical in capturing the heterogenicity of HFpEF pathophysiology. In the present study we utilized a systemic, multi-organ approach by subjecting the whole animal to hypertension and a high fat, high salt WD resulting in a spectrum of pathologies involving a number of organs. Moreover, the diverse spectrum of HFpEF pathologies encompassed in this Gottingen minipig HFpEF model is ideally suited for evaluating the degree to which each of the organ systems is altered and the relative impact of each comorbidity on the overall clinical condition(38).

Until now, absence of an animal model that comprehensively exhibited the heterogenicity of the HFpEF clinical condition forcing the field to compromise and reluctantly accepted animal models that only recapitulated partial HFpEF phenotypes(38). Developing a disease phenotypically characterized by multi-comorbidities and multi-organ dysfunction, is further complicated in that each comorbidity is in itself a stand-alone multifaceted disease with distinct phenotypes driven by a combination of different genetic, dietary and environmental factors. The challenge, similar to that observed among patients with HFpEF, is that with a higher burden of comorbidities comes a higher risk of mortality(5). So too, high mortality rates have been reported in many preclinical HF swine models induced using multiple insults to mimic the multiple comorbidities (21) as had occurred during our initial development of the model. Early in model development, we failed to recognize the unique impact of the Gottingen minipig strain on disease progression. Reliance on previously reported doses and duration of DOCA administration using younger Landrace swine(20,46,47), proved catastrophic causing circulating lipid levels to soar, systemic and LV pressures to skyrocket resulting in rapid end-organ damage and circulatory collapse.

The “too much, too fast” is certainly not the clinical scenario as aging in the setting of long-standing hypertension and metabolic syndrome are critically important contributors to the HFpEF condition. Gottingen miniswine are commonly used as models of obesity, metabolic syndrome and hypercholesterolemia(28-30) manifesting as early as after 2-5 weeks of high-cholesterol/high-fat diet in comparison to most other metabolic syndrome swine models including the Ossabaw strain where features of this disorder require 3-6 months of diet to appear. The applicability of using the Gottingen minipig strain over other swine strains for modeling human HFpEF is further evidenced by its genetic diversity. It has been suggested that similar to human HFpEF, the use of outbred murine colonies could contribute to a better experimental setting because, in contrast, the use of inbred strains represents limited genetic diversity and might not reflect the responses generated in a diverse human population(1,48). Though the Ossabaw strain encompasses descendants of minipigs brought from Spain, their isolation on Ossabaw Island has resulted in a breed that has lived in relative genetic isolation for centuries(49). Gottingen minipig genetic diversity stems from the crossbreeding the Minnesota minipig, the Vietnamese pot-bellied pig, and the German Landrace pig(28). Lastly, and equally important when developing animal models designed to assess therapeutic efficacy of interventions, body size often limits the type of study. The major advantage of rodents is the fact that pharmacological studies require lesser amounts of test agents and rodents are suitable for genetic manipulations to create transgenic models that aid in the elucidation of pathological mechanisms. However, given the current lack of effective treatments for HFpEF coupled with the multifactorial nature of HFpEF pathophysiology, devices and technologies are also being explored thereby necessitating animals large enough as would be deployed in HFpEF patients. The size of the Gottingen minipig makes this strain more suitable for HFpEF studies in that they are the smallest of the swine breeds thereby facilitating pharmacological studies while at the same time large enough for device testing.

### CONCLUSIONS

In summary, our results demonstrate that the combination of DOCA-salt induced hypertension and a high salt, WD-induced metabolic syndrome in the Gottingen minipig strain exhibit a full spectrum of HFpEF phenotypes. This large animal model of HFpEF represents an important preclinical research tool that will drive future studies to identify key molecular mechanisms and evaluate potential therapeutics for HFpEF. The Gottingen minipig HFpEF model also permits a multidimensional phenotypic readout of therapeutic efficacy (i.e. antihypertensive effects, obesity and metabolic syndrome effects), which can help identify patient subgroups most likely to benefit from a specific intervention.

### PERSPECTIVES

Clinical Competencies—The increasing prevalence of heart failure with preserved ejection fraction (HFpEF) coupled with a lack of effective therapies represents a unmet need in cardiovascular medicine. The heterogeneity of the syndrome has created a challenging task to pinpoint the underlying mechanism which drive human HFpEF. This is further exacerbated by a lack of preclinical models which effectively recapitulate the clinical scenario. In order to better understand the pathological drivers of HFpEF manifestation and progress, new preclinical models must be developed.

Translational Outlook—The lack of preclinical animal models that encompass the systemic, multi-organ dysfunction and comorbid-laden phenotype observed in patients has led to an inability to tease out mechanistic insights and test new therapeutic strategies. Herein, we have developed a large animal model integrating multiple co-morbid determinants of HFpEF in a miniswine breed that exhibits sensitivity to obesity, metabolic syndrome and vascular disease with overt clinical signs of heart failure. This model will allow for identification of key mechanisms and testing of new therapeutic strategies thereby permitting better clinical translation.

### REFERENCES CITED IN THIS EXAMPLE

1. Shah S J, Borlaug B A, Kitzman D W et al. Research Priorities for Heart Failure With Preserved Ejection Fraction. Circulation 2020; 141:1001-1026.
2. Zile M R, Gaasch W H, Carroll J D et al. Heart failure with a normal ejection fraction: is measurement of diastolic function necessary to make the diagnosis of diastolic heart failure? Circulation 2001; 104:779-82.
3. Shah S J, Katz D H, Deo R C. Phenotypic spectrum of heart failure with preserved ejection fraction. Heart Fail Clin 2014; 10:407-18.
4. Lourenco A P, Leite-Moreira A F, Balligand J L et al. An integrative translational approach to study heart failure with preserved ejection fraction: a position paper from the Working Group on Myocardial Function of the European Society of Cardiology. Eur J Heart Fail 2018; 20:216-227.
5. Pandey A, Vaduganathan M, Arora S et al. Temporal Trends in Prevalence & Prognostic Implications of Comorbidities Among Patients with Acute Decompensated Heart Failure: The ARIC Study Community Surveillance. Circulation 2020.
6. Hamdani N, Franssen C, Lourenco A et al. Myocardial Titin Hypophosphorylation Importantly Contributes to Heart Failure With Preserved Ejection Fraction in a Rat Metabolic Risk Model. Circulation: Heart Failure 2013; 6:1239-1249.
7. Schauer A, Draskowski R, Jannasch A et al. ZSF1 rat as animal model for HFpEF: Development of reduced diastolic function and skeletal muscle dysfunction. ESC Heart Failure 2020; 7:2123-2134.
8. He K L, Dickstein M, Sabbah H N et al. Mechanisms of heart failure with well preserved ejection fraction in dogs following limited coronary microembolization. Cardiovasc Res 2004; 64:72-83.
9. Lin J, Zhu X, Chade A R et al. Monocyte chemoattractant proteins mediate myocardial microvascular dysfunction in swine renovascular hypertension. Arterioscler Thromb Vasc Biol 2009; 29:1810-6.
10. Marshall K D, Muller B N, Krenz M et al. Heart failure with preserved ejection fraction: chronic low-intensity interval exercise training preserves myocardial 02 balance and diastolic function. J Appl Physiol (1985) 2013; 114:131-47.
11. Ishikawa K, Aguero J, Oh J G et al. Increased stiffness is the major early abnormality in a pig model of severe aortic stenosis and predisposes to congestive heart failure in the absence of systolic dysfunction. J Am Heart Assoc 2015; 4.
12. Yarbrough W M, Mukherjee R, Stroud R E et al. Progressive induction of left ventricular pressure overload in a large animal model elicits myocardial remodeling and a unique matrix signature. J Thorac Cardiovasc Surg 2012; 143:215-23.
13. Charles C J, Lee P, Li R R et al. A porcine model of heart failure with preserved ejection fraction: magnetic resonance imaging and metabolic energetics. ESC Heart Fail 2020; 7:92-102.
14. Gyöngyösi M, Pavo N, Lukovic D et al. Porcine model of progressive cardiac hypertrophy and fibrosis with secondary postcapillary pulmonary hypertension. J Transl Med 2017; 15:202.
15. Kawase Y, Ly H Q, Prunier F et al. Reversal of Cardiac Dysfunction After Long-Term Expression of SERCA2a by Gene Transfer in a Pre-Clinical Model of Heart Failure. Journal of the American College of Cardiology 2008; 51:1112-1119.
16. Klein F J, Bell S, Runte K E et al. Heart rate-induced modifications of concentric left ventricular hypertrophy: exploration of a novel therapeutic concept. Am J Physiol Heart Circ Physiol 2016; 311:H1031-H1039.
17. Rienzo M, Bizé A, Pongas D et al. Impaired left ventricular function in the presence of preserved ejection in chronic hypertensive conscious pigs. Basic Res Cardiol 2012; 107:298.
18. Weil B R, Techiryan G, Suzuki G, Konecny F, Canty J M. Adaptive Reductions in Left Ventricular Diastolic Compliance Protect the Heart From Stretch-Induced Stunning. JACC: Basic to Translational Science 2019; 4:527-541.
19. Zhang N, Feng B, Ma X, Sun K, Xu G, Zhou Y. Dapagliflozin improves left ventricular remodeling and aorta sympathetic tone in a pig model of heart failure with preserved ejection fraction. Cardiovascular Diabetology 2019; 18:107.
20. Schwarzl M, Hamdani N, Seiler S et al. A porcine model of hypertensive cardiomyopathy: implications for heart failure with preserved ejection fraction. Am J Physiol Heart Circ Physiol 2015; 309:H1407-18.
21. Sorop O, Heinonen I, van Kranenburg M et al. Multiple common comorbidities produce left ventricular diastolic dysfunction associated with coronary microvascular dysfunction, oxidative stress, and myocardial stiffening. Cardiovascular Research 2018; 114:954-964.
22. Borlaug B A, Carter R E, Melenovsky V et al. Percutaneous Pericardial Resection: A Novel Potential Treatment for Heart Failure With Preserved Ejection Fraction. Circ Heart Fail 2017; 10: e003612.
23. van den Dorpel M M P, Heinonen I, Snelder S M et al. Early detection of left ventricular diastolic dysfunction using conventional and speckle tracking echocardiography in a large animal model of metabolic dysfunction. Int J Cardiovasc Imaging 2018; 34:743-749.
24. Olver T D, Edwards J C, Jurrissen T J et al. Western Diet-Fed, Aortic-Banded Ossabaw Swine: A Preclinical Model of Cardio-Metabolic Heart Failure. JACC Basic to translational science 2019; 4:404-421.
25. von Scheidt M, Zhao Y, Kurt Z et al. Applications and Limitations of Mouse Models for Understanding Human Atherosclerosis. Cell Metabolism 2017; 25:248-261.
26. Neeb Z P, Edwards J M, Alloosh M, Long X, Mokelke E A, Sturek M. Metabolic syndrome and coronary artery disease in Ossabaw compared with Yucatan swine. Comparative Medicine 2010; 60:300-315.
27. Hand M S, Surwit R S, Rodin J, Van Order P, Feinglos M N. Failure of genetically selected miniature swine to model NIDDM. Diabetes 1987; 36:284-7.
28. Johansen T, Hansen H S, Richelsen B, Malmlöf K. The Obese Gottingen Minipig as a Model of the Metabolic Syndrome: Dietary Effects on Obesity, Insulin Sensitivity, and Growth Hormone Profile. Comparative Medicine 2001; 51:150-155.
29. Ludvigsen T P, Kirk R K, Christoffersen B Ø et al. Gottingen minipig model of diet-induced atherosclerosis: influence of mild streptozotocin-induced diabetes on lesion severity and markers of inflammation evaluated in obese, obese and diabetic, and lean control animals. Journal of Translational Medicine 2015; 13:312.
30. Renner S, Blutke A, Dobenecker B et al. Metabolic syndrome and extensive adipose tissue inflammation in morbidly obese Gottingen minipigs. Molecular Metabolism 2018; 16:180-190.
31. Segal J B, McNamara R L, Miller M R et al. The evidence regarding the drugs used for ventricular rate control. J Fam Pract 2000; 49:47-59.
32. Sharp T E, Polhemus D J, Li Z et al. Renal Denervation Prevents Heart Failure Progression Via Inhibition of the Renin-Angiotensin System. Journal of the American College of Cardiology 2018; 72:2609-2621.
33. Douglas P S, Carabello B A, Lang R M et al. 2019 ACC/AHA/ASE Key Data Elements and Definitions for Transthoracic Echocardiography: A Report of the American College of Cardiology/American Heart Association Task Force on Clinical Data Standards (Writing Committee to Develop Cardiovascular Endpoints Data Standards) and the American Society of Echocardiography. J Am Coll Cardiol 2019; 74:403-469.
34. Sharp T E, Gong Z, Scarborough A et al. Efficacy of a Novel Mitochondrial-Derived Peptide in a Porcine Model of Myocardial Ischemia/Reperfusion Injury. JACC: Basic to Translational Science 2020:466.
35. Donnarumma E, Ali M J, Rushing A M et al. Zofenopril Protects Against Myocardial Ischemia-Reperfusion Injury by Increasing Nitric Oxide and Hydrogen Sulfide Bioavailability. J Am Heart Assoc 2016; 5.
36. Bradley J M, Islam K N, Polhemus D J et al. Sustained release nitrite therapy results in myocardial protection in a porcine model of metabolic syndrome with peripheral vascular disease. Am J Physiol Heart Circ Physiol 2015; 309:H305-17.
37. Böhm F, Pernow J. The importance of endothelin-1 for vascular dysfunction in cardiovascular disease. Cardiovascular Research 2007; 76:8-18.
38. Roh J, Houstis N, Rosenzweig A. Why Don't We Have Proven Treatments for HFpEF? Circ Res 2017; 120:1243-1245.
39. Conceição G, Heinonen I, Lourenço A P, Duncker D J, Falcão-Pires I. Animal models of heart failure with preserved ejection fraction. Neth Heart J 2016; 24:275-86.
40. Paulus W J, Tschope C, Sanderson J E et al. How to diagnose diastolic heart failure: a consensus statement on the diagnosis of heart failure with normal left ventricular ejection fraction by the Heart Failure and Echocardiography Associations of the European Society of Cardiology. Eur Heart J 2007; 28:2539-50.
41. Borlaug B A, Paulus W J. Heart failure with preserved ejection fraction: pathophysiology, diagnosis, and treatment. Eur Heart J 2011; 32:670-9.
42. Pieske B, Tschöpe C, de Boer R A et al. How to diagnose heart failure with preserved ejection fraction: the HFA-PEFF diagnostic algorithm: a consensus recommendation from the Heart Failure Association (HFA) of the European Society of Cardiology (ESC). Eur J Heart Fail 2020; 22:391-412.
43. Terris J M, Berecek K H, Cohen E L, Stanley J C, Whitehouse W M, Jr., Bohr D F. Deoxycorticosterone hypertension in the pig. Clin Sci Mol Med Suppl 1976; 3:303s-305s.
44. Heinonen I, Sorop O, van Dalen B M et al. Cellular, mitochondrial and molecular alterations associate with early left ventricular diastolic dysfunction in a porcine model of diabetic metabolic derangement. Scientific Reports 2020; 10:13173.
45. Reddy Y N V, Carter R E, Obokata M, Redfield M M, Borlaug B A. A Simple, Evidence-Based Approach to Help Guide Diagnosis of Heart Failure With Preserved Ejection Fraction. Circulation 2018; 138:861-870.
46. Ciccone C D, Zambraski E J. Renal function in two-kidney deoxycorticosterone acetate-hypertensive Yucatan miniature swine. Proc Soc Exp Biol Med 1981; 168:218-21.
47. Zhang X, Lerman L O. Investigating the Metabolic Syndrome: Contributions of Swine Models. Toxicol Pathol 2016; 44:358-66.
48. Valero-Munoz M, Backman W, Sam F. Murine Models of Heart Failure with Preserved Ejection Fraction: a “Fishing Expedition”. JACC Basic to translational science 2017; 2:770-789.
49. Martin R J, Gobble J L, Hartsock T H, Graves H B, Ziegler J H. Characterization of an obese syndrome in the pig. Proc Soc Exp Biol Med 1973; 143:198-203.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed is:

1. An animal HFpEF composition comprising:
a hypertensive agent formulated in a dose of about 5 mg/kg to about 100 mg/kg; and
an animal chow comprising about a 50%-50% (wt/wt) proportion of standard diet and custom diet.

2. The HFpEF composition of claim 1, wherein the hypertensive agent comprises a pellet or continuous infusion.

3. The HFpEF composition of claim 1, wherein the hypertensive agent comprises 11-deoxycorticosterone acetate (DOCA), N(gamma)-nitro-L-arginine methyl ester (L-NAME), or Angiotensin II (ANG II).

4. The HFpEF composition of claim 1, wherein the standard diet comprises about 33% carbohydrate, about 3.9% fat, about 16.1% protein, about 0% cholesterol, about 0.4% salt, about 2.3 kcal/g energy density, or a combination thereof.

5. The HFpEF composition of claim 1, wherein the custom diet comprises about 39.8% carbohydrate, about 40% to about 44.1% fat, about 16.2% protein, about 2% cholesterol, about 0.5% to about 0.7% sodium cholate, about 4.01 kcal/g energy density, or a combination thereof.

6. The HFpEF composition of claim 5, wherein the custom diet further comprises about 4% salt.

7. The HFpEF composition of claim 5, wherein carbohydrates comprise about 17.8% fructose.

8. The HFpEF composition of claim 3, wherein the DOCA is formulated in a dose of about 25 mg/kg to about 100 mg/kg.

9. A method for inducing heart failure with preserved ejection fraction (HFpEF) in a laboratory animal, the method comprising administering the HFpEF composition of any one of claims 1-8 to a swine laboratory animal.

10. A method for inducing heart failure with preserved ejection fraction (HFpEF) in a laboratory animal, the method comprising administering to an animal an HFpEF composition comprising:
a hypertensive agent formulated in a dose of about 5 mg/kg to about 100 mg/kg, wherein the hypertensive agent comprises 11-deoxycorticosterone acetate (DOCA), N(gamma)-nitro-L-arginine methyl ester (L-NAME), or Angiotensin II (ANG II), and
an animal chow comprising about a 50%-50% (wt/wt) proportion of standard diet and custom diet, wherein the standard diet comprises about 33% carbohydrate, about 3.9% fat, about 16.1% protein, about 0% cholesterol, about 0.4% salt, about 2.3 kcal/g energy density, or a combination thereof, wherein the custom diet comprises about 39.8% carbohydrate, about 40% to about 44.1% fat, about 16.2% protein, about 2% cholesterol, about 0.5% to about 0.7% sodium cholate, about 4.01 kcal/g energy density, or a combination thereof.

11. The method of claim 10, wherein the hypertensive agent comprises a pellet or continuous infusion.

12. The method of claim 10, wherein DOCA is formulated in a dose of about 25 mg/kg to about 100 mg/kg.

13. The method of claim 10, wherein the custom diet further comprises about 4% salt.

14. The method of claim 10, wherein carbohydrates of the custom diet comprises about 17.8% fructose.

15. The method of claim 9 or 10, wherein the administering occurs from 30 days to 90 days to induce and maintain high blood pressure.

16. The method of claim 15, wherein the blood pressure comprises an arterial systolic blood pressure ≥130 mmHg, an arterial diastolic blood pressure ≥90 mmHg, a mean arterial blood pressure ≥100 mmHg, a left ventricular end-diastolic pressure ≥14 mmHg, or a combination thereof.

17. The method of claim 9 or 10, wherein the DOCA pellet is administered subcutaneously or orally.

18. The method of claim 9 or 10, wherein DOCA, L-NAME, or ANG II is administered subcutaneously, intravenously, transdermal, or orally.

19. The method of claim 9 or 10, wherein the animal chow is provided daily as a total dry weight of about 900 grams.

20. The method of claim 9 or 10 further comprising providing water ad libitum to the animal.

21. The method of claim 20, wherein the water comprises about 40 mg salt per 1 liter.

22. The method of claim 20, wherein the animal chow and water are administered for at least 20 weeks.

23. The method of claim 9 or 10, wherein the animal is a swine laboratory animal.

24. The method of claim 23, wherein the swine animal is a Gottingen Minipig®.

25. A method of treating a swine to cause it to have heart failure with preserved ejection (HFpEF), whereby the swine can be used as an animal model, the method comprising:
administering to a swine an animal HFpEF composition, wherein the HFpEF composition comprises
i. a hypertensive agent formulated in a dose of about 5 mg/kg to about 100 mg/kg, wherein the hypertensive agent comprises 11-deoxycorticosterone acetate (DOCA), N(gamma)-nitro-L-arginine methyl ester (L-NAME), or Angiotensin II (ANG II), and
ii. an animal chow comprising about a 50%-50% (wt/wt) proportion of standard diet and custom diet, wherein the standard diet comprises about 33% carbohydrate, about 3.9% fat, about 16.1% protein, about 0% cholesterol, about 0.4% salt, about 2.3 kcal/g energy density, or a combination thereof, wherein the custom diet comprises about 39.8% carbohydrate, about 40% to about 44.1% fat, about 16.2% protein, about 2% cholesterol, about 0.5% to about 0.7% sodium cholate, about 4.01 kcal/g energy density, or a combination thereof,
inducing high blood pressure above an arterial systolic blood pressure of about 150 mmHG in the swine by 30 days; and
maintaining high blood pressure above an arterial systolic blood pressure of about 150 mmHG in the swine for at least 30 days.

26. The method of claim 25, wherein the agent comprises a pellet comprising DOCA formulated in a dose of about 25 mg/kg to about 100 mg/kg.

27. The method of claim 25, further comprising increasing circulating LDL cholesterol levels above about 150 mg/DL for 30 days.

28. The method of claim 25, wherein the custom diet further comprises about 4% salt.

29. The method of claim 25, wherein the carbohydrates of the custom diet comprises about 17.8% fructose.

30. The method of claim 25, wherein the blood pressure comprises an arterial systolic blood pressure ≥about 130 mmHg, an arterial diastolic blood pressure ≥about 90 mmHg, a mean arterial blood pressure ≥about 100 mmHg, or a combination thereof.

31. The method of claim 25, wherein the blood pressure comprises an arterial systolic blood pressure ≥about 150 mmHg, an arterial diastolic blood pressure ≥about 110 mmHg, a mean arterial blood pressure ≥about 130 mmHg, or a combination thereof.

32. The method of claim 19, wherein the DOCA pellet is administered subcutaneously or orally.

33. The method of claim 25, wherein DOCA, L-NAME, or ANG II is administered subcutaneously, intravenously, transdermal, or orally.

34. The method of claim 25, wherein the animal chow is provided daily as a total dry weight of about 900 grams.

35. The method of claim 25 further comprising providing water ad libitum to the animal.

36. The method of claim 35, wherein the water comprises about 40 mg salt per 1 liter.

37. The method of claim 35, wherein the animal chow and water are administered for at least 20 weeks.

38. The method of claim 25, wherein the swine animal is a Gottingen Minipig®.

39. A heart failure with preserved ejection (HFpEF) animal model prepared by the method of claim 9, 10, or 25.

40. A kit for a heart failure with preserved ejection (HFpEF) animal model, the kit comprising:
an animal HFpEF composition comprising
a hypertensive agent formulated in a dose of about 5 mg/kg to about 100 mg/kg, wherein the hypertensive agent comprises 11-deoxycorticosterone acetate (DOCA), N(gamma)-nitro-L-arginine methyl ester (L-NAME), or Angiotensin II (ANG II), and
an animal chow comprising about a 50%-50% (wt/wt) proportion of standard diet and custom diet, wherein the standard diet comprises about 33% carbohydrate, about 3.9% fat, about 16.1% protein, about 0% cholesterol, about 0.4% salt, about 2.3 kcal/g energy density, or a combination thereof, wherein the custom diet comprises about 39.8% carbohydrate, about 40% to about 44.1% fat, about 16.2% protein, about 2% cholesterol, about 0.5% to about 0.7% sodium cholate, about 4.01 kcal/g energy density, or a combination thereof; and
a Gottingen Minipig®

41. The kit of claim 40, wherein the agent comprises a pellet comprising DOCA formulated in a dose of about 25 mg/kg to about 100 mg/kg.
